(19) United States
(12) Patent Application Publication
(10) Pub. No.: US 2024/0156756 A1
(43) Pub. Date: May 16, 2024

(54) TOPICAL WIPE CONTAINING NIFEDIPINE AND LIDOCAINE

(71) Applicant: Steel Therapeutics, Inc., Iowa City, IA (US)

(72) Inventors: Matthew Stahl, Jesup, IA (US); Robbie Schwenker, North Liberty, IA (US)

(21) Appl. No.: 18/375,115

(22) Filed: Sep. 29, 2023

Related U.S. Application Data
(63) Continuation of application No. PCT/US2022/023130, filed on Apr. 1, 2022.
(60) Provisional application No. 63/169,768, filed on Apr. 1, 2021.

(51) Int. Cl. A61K 31/167; A61K 9/00; A61K 9/70; A61K 31/4422

(52) U.S. Cl. CPC A61K 31/167; A61K 9/0014; A61K 9/7007; A61K 31/4422

(57) ABSTRACT

The present disclosure provides, amongst other things, pharmaceutical compositions, kits and methods for treatment of anal fissures using a medicated topical wipe comprising Nifedipine and Lidocaine.

### CROSS REFERENCE

This application is a continuation of International Patent Application No. PCT/US2022/023130, filed Apr. 1, 2022, which claims priority under 35 U.S.C. § 119(e) to U.S. Provisional Patent Application No. 63/169,768 filed Apr. 1, 2021, entitled “Topical Wipe Containing Nifedipine and Lidocaine”, the contents of each of which is hereby incorporated by reference in its entirety herein.

### FIELD OF THE INVENTION

The present invention is related to the field of treating soft tissue injuries, and more particularly to a topical wipe for the delivery of therapeutic compounds in the treatment of anal fissures.

### BACKGROUND

The organs and soft tissues of the human body, generally defined as the tissues not hardened by ossification or calcification, comprise the bulk of the composition of the human body. Such tissues include muscles, tendons, ligaments, fat, skin, and the like. Soft tissues are generally very elastic materials, capable of experiencing significant deformation without failure, return to their natural configuration. Regardless, under certain forces and loads, even the soft tissues experience failure and may tear. While many such tears are mild and heal naturally over time, some are more serious and can result in discomfort and pain. Additionally, some tears in sensitive locations are particularly painful and can inhibit day-to-day life activities. Soft tissue damage in certain parts of the body is also embarrassing or difficult for patients to discuss due to taboos and other social attitudes.

One such example is anal fissures, generally defined as small tears in the tissue that lines the anus. Although such tears are most common in infants, they can happen to people of all ages and occur in about 1 in 350 adults. Like most soft tissue damage, shallow or superficial fissures usually heal naturally on their own, and the associated discomfort can sometimes be mitigated, and healing accelerated, by simple treatments. However, in some cases, serious cases may not heal naturally and require medication or surgery.

Medications such as nitroglycerine ointment and calcium channel blockers such as nifedipine, diltiazem and indoramin have been used for treatment of anal fissures. Golfam et al. (Acta medica Iranica, Vol. 48, No. 5, 2010, pages 295-299) treated chronic anal fissures with a cream containing 0.5% nifedipine for four weeks and found that 70% of patients experienced healing. Wise (U.S. Pat. No. 10,543,201) treats the internal anal sphincter and/or the pelvic floor with a topical composition containing 0.3% nifedipine. The internal anal sphincter is located deep inside the anal canal (more than 1 cm beyond the anal canal) and one cannot apply topical composition to that tissue without the use of an applicator device or by digital deep penetration. Gels or creams are typically applied digitally by the patient or with a syringe or suppository. The patient covers the finger with a plastic wrap or disposable glove, squeezes the prescribed amount of ointment onto the covered finger, and then digitally spreads the ointment on the affected area. This type of application is not always practical or comfortable. Some patients lack the flexibility or dexterity to apply the ointment in this fashion. Those patients who can apply the ointment often find the process uncomfortable. This can lead to improper or incomplete application, which can limit the effectiveness of the treatment, or discourage patients from applying the treatment at all.

### SUMMARY OF EMBODIMENTS

In one aspect, the disclosure provides a topical wipe comprising a fibrous substrate material moistened with a solution comprising 0.15% to 7.5% w/w lidocaine and salts thereof (example Lidocaine HCl) by concentration and 0.15 to 1.5% w/w nifedipine and salts thereof by concentration.

In a related aspect, the disclosure provides a topical wipe comprising a fibrous substrate material moistened with a solution comprising 1.5% to 7.5% w/w lidocaine and salts thereof (example Lidocaine HCl) by concentration and 0.3 to 1.5% w/w nifedipine and salts thereof by concentration.

In a related aspect, the disclosure provides a topical wipe comprising a fibrous substrate material moistened with a solution comprising 0.15%, or 0.2%, or 0.25%, or 0.3%, or 0.35%, or 0.4%, or 0.45%, or 0.5%, or 0.8%, or 1%, or 1.2% or 1.5%, or 1.8%, or 2%, or 2.2%, or 2.5%, or 2.8%, or 3%, or 3.2%, or 3.5%, or 3.8%, or 4% or 4.2%, or 4.5% or 4.8%, or 5%, or 5.2%, or 5.4%, or 5.6%, or 5.8%, or 6%, or 6.5%, or 6.8%, or 7% or 7.5% w/w lidocaine and salts thereof (example Lidocaine HCl) by concentration and 0.15%, or 0.18%, or 0.2%, or 0.22%, or 0.25%, or 0.28%, or 0.3%, or 0.32%, or 0.35%, or 0.38%, or 0.4%, or 0.42%, or 0.45% or 0.5%, or 0.55%, or 0.6%, or 0.65%, or 0.7%, or 0.75%, or 0.80%, or 0.85%, or 0.90%, or 0.95%, or 1%, or 1.2%, or 1.5% w/w nifedipine and salts thereof by concentration.

In a related aspect, the disclosure provides a topical wipe comprising a fibrous substrate material moistened with a solution comprising 1.5%, or 1.8%, or 2%, or 2.2%, or 2.5%, or 2.8%, or 3%, or 3.2%, or 3.5%, or 3.8%, or 4% or 4.2%, or 4.5% w/w lidocaine and salts thereof (example Lidocaine HCl) by concentration and 0.15%, or 0.18%, or 0.2%, or 0.22%, or 0.25%, or 0.28%, or 0.3%, or 0.32%, or 0.35%, or 0.38%, or 0.4%, or 0.42%, or 0.45% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.2% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.1% nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.15% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.25% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.35% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.4% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.45% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.5% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 1% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 1.5% w/w nifedipine and salts thereof by concentration.

In some embodiments, the disclosure provides a topical wipe comprising a fibrous substrate material moistened with a solution comprising at least 0.15% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 0.2% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 0.4% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 0.6% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 0.8% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 1% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 1.5% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 2% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 2.5% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 3% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 4% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 4.5% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 5% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 6% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 6.5% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 7% w/w lidocaine and salts thereof (example Lidocaine HCl) by concentration and at least 0.3% w/w nifedipine and salts thereof by concentration.

In some embodiments, the disclosure provides a topical wipe comprising a fibrous substrate material moistened with a solution comprising at least 7.5% w/w lidocaine and salts thereof (example Lidocaine HCl) by concentration and at least 0.3% w/w nifedipine and salts thereof by concentration.

In some embodiments of any of the wipes described herein, the topical wipe comprising a fibrous substrate material moistened with a solution comprising about 0.15-7.5% w/w lidocaine and salts thereof (example Lidocaine HCl) by concentration and about 0.3 to 1.5% w/w nifedipine and salts thereof by concentration is capable of treating anal fissures.

In some embodiments of any of the wipes described herein, the topical wipe comprising a fibrous substrate material moistened with a solution comprising about 1.5-5% w/w lidocaine and salts thereof (example Lidocaine HCl) by concentration and about 0.15 to 0.45% w/w nifedipine and salts thereof by concentration is capable of treating anal fissures.

In some embodiments of any of the wipes described herein, the topical wipe further comprises a preservative. In some embodiments, the topical wipe further comprises an antioxidant. In some embodiments, the topical wipe further comprises at least one excipient. In some embodiments, the excipient is selected from the group consisting of: excipient is selected from the group consisting of: antioxidants, penetration enhancers, preservatives, moisture retainers, dispersing agents, humectant, emulsifier, plasticizer, surfactant, viscosity modifiers, emollients, film forming agents, tailing solvents, co-solvents, and/or oils. In some embodiments, the topical wipe further comprises an alcohol solvent.

In some embodiments of any of the wipes described herein, further comprises penetration enhancer, said penetration enhancer is selected from the group consisting of benzyl alcohol, dimethyl isosorbide (DMI), propylene glycol, hexylene glycol, isopropyl alcohol, ethanol, phenoxyethanol, oleic acid, olelyl alcohol, isopropyl myrisitate, medium chain triglyceride (MCT) and transcutol.

In some embodiments of any of the wipes described herein, further comprises humectant, said humectant is selected from the group consisting of propylene carbonate, glycerin, pentylene glycol, butylene glycol, aloe vera juice, extract, hexylene glycol, hyaluronic acid and lactic acid.

In some embodiments of any of the wipes described herein, further comprises emulsifier, said emulsifier is selected from the group consisting of Polysorbate (20 to 80), Span-80, PEG-40, hydrogenated castor oil, sodium lauryl sulfate (SLS), poloxamers, sorbitans (20-85) and glyceryl monooleate (GMO).

In some embodiments of any of the wipes described herein, further comprises surfactant, said surfactant is selected from the group consisting of PEG 400, tween-80, oleyl alcohol, glycerin, hexylene glycol and propylene glycol.

In some embodiments of any of the wipes described herein, further comprises antioxidant, said antioxidant is selected from the group consisting of butylated hydroxytoluene (BHT), alpha-tocopherol, propyl gallate, ascorbic acid squalene, ascorbyl palmitate, sodium thiosulfate and sodium metabisulphate.

In some embodiments of any of the wipes described herein, further comprises bio-adhesive polymer, said polymer is selected from the group consisting of carbomer homopolymer Type A, B and C, carbomer copolymers, interpolymers, polycarbophils, pemulens, hydroxyethyl cellulose (HEC), hydroxypropyl cellulose (HPC) and hydroxypropyl methylcellulose (HPMC).

In some embodiments of any of the wipes described herein, the solution comprises a solvent selected from the group consisting of water, propylene glycol and hexylene glycol.

In some embodiments of any of the wipes described herein, said wipe further comprises one or more of benzyl alcohol, transcutol, propylene carbonate, polysorbate 80, BHT, PEG 400, glycerin and hexylene glycol.

In some embodiments of any of the wipes described herein, said wipe comprises at least 2% benzyl alcohol by concentration.

In some embodiments of any of the wipes described herein, said wipe comprises 30 to 40% transcutol by concentration, particularly 30%, 35% or 40% transcutol by concentration.

In some embodiments of any of the wipes described herein, said wipe comprises at least 4% propylene carbonate by concentration.

In some embodiments of any of the wipes described herein, said wipe comprises at least 2% polysorbate 80 by concentration.

In some embodiments of any of the wipes described herein, said wipe comprises at least 0.2% BHT by concentration.

In some embodiments of any of the wipes described herein, said wipe comprises 30 to 60% propylene glycol by concentration, particularly 30%, 35%, 40%, 45%, 50%, 55%, or 60% propylene glycol by concentration.

In some embodiments of any of the wipes described herein, said wipe comprises at least 15% glycerin by concentration.

In some embodiments of any of the wipes described herein, said wipe comprises at least 10% hexylene glycol by concentration.

In some embodiments of any of the wipes described herein, wipe comprises formulation selected from the group consisting of formulation I, formulation II, formulation III, formulation IV, formulation V, and formulation VI.

In some embodiments of any of the wipes described herein, the topical wipe is made from a fiber. In some embodiments, the topical wipe is made from a non-woven fiber. In some embodiments, the topical wipe is made from a woven fiber.

In some embodiments of any of the wipes described herein, the topical wipe is in a generally rectangular shape. In some embodiments, the topical wipe is individually packaged for one-time use.

In another aspect, the disclosure provides a container comprising a plurality of topical wipes described herein. In some embodiments, the container comprises a soft pack. In some embodiments, the container comprises a hard pack.

In another aspect, the disclosure provides a kit comprising a plurality of topical wipes described herein in separate individual single use packs or in bulk of at least 30 wipes each.

In another aspect, the disclosure provides a method of treating or ameliorating anal fissures in a subject in need thereof, comprising the step of applying the topical wipe comprising a fibrous substrate material moistened with a solution comprising at least 1.5% lidocaine HCl by concentration and at least 0.3% nifedipine by concentration to the perianal area of said subject thereby providing therapeutic relief.

In another aspect, the disclosure provides a method of treating or ameliorating anal fissures in a subject in need thereof, comprising the step of applying the formulation comprising 1.5% to 5% lidocaine HCl, preferably at least 1.5% lidocaine HCl by concentration and 0.15-4.5% nifedipine, preferably at least 0.15% nifedipine by concentration to anal tissue within 1 cm from the anal canal of said subject thereby providing therapeutic relief.

In another aspect, the disclosure provides a method of treating or ameliorating anal fissures in a subject in need thereof, comprising the step of applying the formulation comprising 1.5-5% lidocaine HCl, preferably at least 1.5% lidocaine HCl by concentration and 0.15-0.45% nifedipine, preferably at least 0.15% nifedipine by concentration to an external anal sphincter of said subject thereby providing therapeutic relief.

In another aspect, the disclosure provides a method of manufacture of topical wipe comprising 0.15-0.45%, preferably at least 0.3% nifedipine by concentration and 1.5-5% lidocaine HCl, preferably at least 1.5% lidocaine HCl by concentration, comprising the steps of: providing a wipe, applying a formulation comprising at least 0.15% nifedipine and at least 1.5% lidocaine HCl, drying said wipe for sufficient time and packaging said wipe in an airtight container or pouch.

In another aspect, the disclosure provides a method of using a topical wipe, wherein said wipe comprises a formulation of 0.15-0.45% nifedipine, preferably at least 0.15% nifedipine and at least 1.5% lidocaine HCl by concentration, comprising the steps of applying the wipe to the perianal region of a subject such that said formulation is in contact with external anal sphincter or anal tissue which is within 1 cm from the anal canal of said subject, wherein said subject has anal fissure.

In some embodiments, the formulation comprising at least 0.15% nifedipine and at least 1.5% lidocaine HCl, preferably 0.3% nifedipine and 1.5% lidocaine, upon application by means of said wipe to the perianal area is in contact with the external anal sphincter.

In some embodiments, the formulation comprising at least 0.15% nifedipine and at least 1.5% lidocaine HCl, preferably 0.3% nifedipine and 1.5% lidocaine, upon application by means of said wipe to the perianal area is in contact with the external anal sphincter and internal anal sphincter.

In some embodiments, the formulation comprising at least 0.15% nifedipine and at least 1.5% lidocaine HCl, preferably 0.3% nifedipine and 1.5% lidocaine, upon application by means of said wipe to the perianal area is in contact with the external anal sphincter but not the internal anal sphincter.

In some embodiments, the formulation comprising at least 0.15% nifedipine and at least 1.5% lidocaine HCl, preferably 0.3% nifedipine and 1.5% lidocaine, upon application by means of said wipe to the perianal area is in contact with anal tissue that is within 1 cm of the anal cavity.

Certain exemplary embodiments will now be described to provide an overall understanding of the compositions of matter (e.g., formulation of nifedipine and lidocaine HCl), and methods of making the same. The present disclosure provides for various ingredients, and/or combinations thereof, to formulate the compositions of matter, and further provides for various steps that can be used to combine the various ingredients, and/or combinations thereof, to arrive at the compositions of matter. Those skilled in the art will understand that the compositions and methods described herein are non-limiting exemplary embodiments and that the scope of the present disclosure is defined solely by the claims. The features illustrated or described in connection with one exemplary embodiment may typically be combined with the features of other embodiments, unless indicated otherwise. For example, a person skilled in the art, in view of the present disclosure, will understand one or more steps that are provided as part of one method for making compositions of matter as provided for herein that can be used in other methods for making compositions of matter as provided for herein or otherwise known in the art. Likewise, a person skilled in the art will understand various steps that can be interchangeable between various methods disclosed herein or otherwise known to those skilled in the art, or otherwise modified in view of various methods disclosed herein or otherwise known to those skilled in the art, without departing from the spirit of the present disclosure. By way of non-limiting example, a person skilled in the art, in view of the present disclosure, will understand that the use of alternative techniques, whether explicitly disclosed in the present application or known by those skilled in the art in view of the present disclosure, typically still maintains the inventive concepts associated with the present disclosure.

In the present disclosure, a number of different terms can be used interchangeably while still being understood by the skilled person. By way of non-limiting example, the phrases “selected from the group consisting of,” “chosen from,” and the like, include mixtures of the specified materials. Where a numerical limit or range is stated herein, the endpoints are included. Also, all values and subranges within a numerical limit or range are specifically included as if explicitly written out. References to an element in the singular is not intended to mean “one and only one” unless specifically stated, but rather “one or more.” Unless specifically stated otherwise, terms such as “some” refer to one or more, and singular terms such as “a,” “an,” and “the” refer to one or more.

### DESCRIPTION

In one aspect, the disclosure relates to methods and compositions for treating anal fissures by providing a medicated wipe comprising a therapeutic agent and a topical anesthetic, capable of being applied to anus, which amongst other things, can accelerate or facilitate treatment, can aid comfort with topical numbing of pain, and can relax local muscles to increase blood flow to the affected area, thereby facilitating the healing of anal fissures.

The wipe provides a number of improvements over the prior art, including an easier-to-use and more comfortable delivery mechanism for the therapeutic compounds and topical anesthetic; a cooling, soothing, and comforting reduction in pain, irritation, and other discomfort; and chemical compositions that effect better, accelerated healing. The wipe also ensures better compliance with application of topical medication which is unlike the ointments of the prior art that are not always practical or comfortable. The prior art ointments are generally dispersed within and/or around the anal cavity, requiring digital insertion, whereas the topical wipe used herein disperses the treatment compounds externally. This aids in comfort and ease-of-use for the patient because the mechanics of the wiping motion mimic that routine toilet hygiene and do not require insertion. This in turn encourages compliance with treatment regimens and improved outcomes. Additionally, the compounds may be safely exposed to skin, and because use of the wipe does not require digital insertion, the patient may forego the use of disposable gloves or other sanitary covering, and simply wash the hands properly after use.

In some embodiments, the active pharmaceutical compounds in said wipe achieves effective permeating profiles with respect to the targeted tissue to provide therapeutic relief. The permeation profile of each compound is based on, among other things, the physical and chemical properties of each compound, the microstructure characteristics of each compound, the dosage load of the topical wipe, and the characteristics of the target tissue, which can vary substantially in different anatomical regions. That is, formulations and prior art products known to be suitable for use in specific regions, such as the scalp, abdomen, or hands, may not be adequate for use with the topical wipe described herein. An additional consideration is aesthetic, in that the compound formulation may be selected or optimized in consideration of attributes such as viscosity or sensorial feel, which may impact the “messiness” of using the product (e.g., an overly saturated, running compound may drip excessively from the wipe before and after use, or transfer an excess of the formulation to the affected area that does not permeate the skin and must be manually removed).

In some embodiments, the topical wipe is treated with one or more topical agents having therapeutic properties effective to relax the external anal sphincter and increase blood flow. By way of example and not limitation, one such family of compound is calcium channel blockers. By way of further example and not limitation, one such calcium channel blockers is nifedipine, C₁₇H₁₈N₂O₆, depicted below:

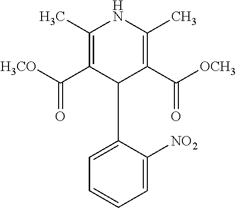 [SMILES: COC(=O)C1=C(C)NC(C)=C(C(=O)OC)C1c1ccccc1[N+](=O)[O-]]

Nifedipine is soluble in organic solvents such as EtOH (3 mg/ml), DMSO (30 mg/ml) and dimethyl formamide (30 mg/ml), which should be purged with an inert gas. One challenge of using nifedipine is that it is sparingly soluble in aqueous buffers, though it is highly lipophilic and soluble in alcohol.

In another embodiment, the topical wipe by also, or alternatively, treated with one or more topical agents having pain- or itch-relieving properties effective to reduce discomfort in the affected area. By way of example and not limitation, one such compound is lidocaine (depicted below) and salts thereof, such as lidocaine HCl, C₁₄H₂₂N₂O, (depicted below):

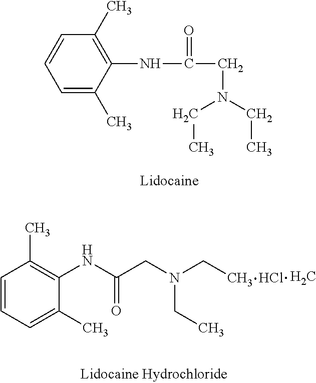

Lidocaine and salts thereof are a local anesthetic with rapid onset of action and is highly soluble in alcohol (4 mg/ml EtOH) and chloroform, and freely soluble in ether, benzene. It also dissolves in oils.

In another embodiment, the topical wipe is pre-moistened with a solution comprising the desired compounds, such as muscle relaxer and/or a pain reliever, and then packaged into a ready-to-dispense form, such as a soft pack or a hard cover container with a slit-top opening that inhibits the introduction of oxygen and other degradants, preserves moisture to inhibit evaporation, and protects the wipes from external contaminants. A wipe may be retrieved from the container for immediate use one at a time as prescribed or needed, and the container then resealed. Alternatively, or additionally, the wipes may be individually packaged in a single-use container or soft packaging.

In some embodiments, the wipe is pre-moistened with a solution comprising about 1.5% w/w lidocaine and about 0.3% w/w nifedipine by concentration. In some embodiments, the wipe is pre-moistened with a solution comprising about 2% w/w lidocaine and about 0.3% w/w nifedipine by concentration. In some embodiments, the wipe is pre-moistened with a solution comprising about 2.5% w/w lidocaine and about 0.3% w/w nifedipine by concentration. In some embodiments, the wipe is pre-moistened with a solution comprising about 3% w/w lidocaine HCl and about 0.3% w/w nifedipine by concentration. In some embodiments, the wipe is pre-moistened with a solution comprising about 3.5% w/w lidocaine and about 0.3% w/w nifedipine by concentration. In some embodiments, the wipe is pre-moistened with a solution comprising about 4% w/w lidocaine and about 0.3% w/w nifedipine by concentration. In some embodiments, the wipe is pre-moistened with a solution comprising about 4.5% w/w lidocaine and about 0.3% w/w nifedipine by concentration. In some embodiments, the wipe is pre-moistened with a solution comprising about 5% w/w lidocaine and about 0.3% w/w nifedipine by concentration. In some embodiments, the wipe is pre-moistened with a solution comprising lidocaine HCl ranging from 1.5 to 5% w/w and about 0.3% w/w nifedipine by concentration.

These concentrations may be altered by the addition of other compounds as discussed elsewhere herein, including but not necessarily limited to preservatives and antioxidants. The concentrations may also vary depending on other factors, such as the quantities and concentrations in which the compounds in question may be synthesized and/or packaged, shipped, and stored in accordance with applicable practical constraints, such as typical prescription quantity, shelf spacing requirements, anticipated sealed shelf-life in common detail environments, shelf-life upon opening and use, and common transportation conditions. In particular, attention should be paid to the introduction of impurities or compound-related degradants that may form during synthesis or storage.

In some embodiments, the topical wipe further comprises suitable excipients and/or solvents, generally selected so as to achieve the desired concentrations. For example, both nifedipine and lidocaine are highly soluble in alcohol. One or more excipients or classes of excipients, including but not necessarily limited to antioxidants, penetration enhancers, moisture retainers, dispersing agents, viscosity modifiers, emollients, film forming agents, preservatives, tailing solvents, co-solvents, oils, and so forth, may be included. The mass and/or volume of the therapeutic compounds used may be altered or adapted to achieve the desired concentration described elsewhere herein in view of the excipients included in any given formulation. Excipients may be selected based on a number of factors, including but not necessarily limited to stability and potential interactions with the therapeutic compounds which would lead to inhibited degradation or other inhibited function at the expected dosage levels.

In some embodiments, the topical wipe comprises a fibrous substrate material, such as a natural or synthetic fiber or cloth, preferably having a smooth, low-friction surface and/or finish that is gentle on sensitive skin. In some embodiments, a non-woven fiber may be used having absorption properties effective to both load the desired combination of drug products, while also having desired storage characteristics (e.g., ability to load and retain the drug compounds in storage for the desired period of shelf-stability without undue decomposition or degradation), use characteristics (resistance to tearing or breakage), and desired disposal characteristics (e.g., biodegradable, compostable, flushable, septic-safe, etc.). In some embodiments, the components of the topical wipe are effective to provide a shelf-stable product for a period of about two years.

In some embodiments, the wipe includes one or more preservatives and/or antioxidants. The class and quantity or concentration of these compounds is preferably effective to ensure the desired shelf-life of the topical wipes under ordinary shipping, retail, and consumer conditions. Techniques for determining appropriate compounds and amounts thereof through routine experimentation and testing are known in the art.

In some embodiments, the topical wipe is generally orthogonal, and preferably rectangular, allowing for ready storage and retrieval. The wipes may be individually packaged, such as in sealed single unit packets or pouches, or may be packed in multiples. The packaging is preferably re-sealable, such as by using zippers or adhesives.

In some embodiments, the topical wipe is frictionless and is gentle on sensitive skin.

In some embodiments, the topical wipe is flushable, disposable, and septic safe.

In some embodiments, the topical wipe is shelf-stable for at least 2 years.

Although the foregoing topical wipe is described with respect to specific compounds for the treatment of anal fissures in humans, it will be readily appreciate that the same considerations and principles are applicable to other areas as well, and may be used to treat similar types of injuries in the tissues of non-human mammals or other animals, and for treating similar types of injuries in other anatomical regions having similar medical properties, anatomical geometries, or social attitudes that inhibit full patient compliance with self-administered therapy.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

### BRIEF DESCRIPTION OF THE DRAWINGS

FIG. 1. shows the process of testing different combinations of excipients and altering concentrations to arrive at formulations of Nifedipine and Lidocaine suitable for impregnating wipes for therapeutic use.

FIG. 2 shows different impregnation methods for creating the topical wipe comprising a formulation of Nifedipine and Lidocaine along with selected excipients. A) Lidocain HCI+Nifedipine solution of required quantity is taken in a container. B) Wipes are dipped or soaked in the active solution for t min. C) Drying of wipes post soaking or dipping for t min. D) Placebo solution of required quantity is taken in a spray bottle. E) Wipes are sprayed with the active solution for t min. F) Drying of wipes post spraying for t min. G) Placebo solution of required quantity is taken in a pipette. H) Wipes are wetted (incremental addition of formulation onto wipes using pipette) with the active solution by pipetting for t min. I) Drying of wipes post pipetting for t min. Saturation Loading is the final weight of wipe/dry weight of wipe); Percentage of saturation is final weight of wipe/dry weight of wipe)×100; and Weight of impregnated formulation is (Weight of dry wipe post loading and drying)−(Tare Weight of Dry Wipe).

FIG. 3 shows the drying method used consistently for drying all the wipes impregnated with different methods.

FIG. 4 shows graphs of weight of impregnated solution, saturation loading and % saturation loading with each incremental addition steps. The wipes were dried for 45 min post each addition step.

FIG. 5 shows graphs of weight of impregnated solution, saturation loading and % saturation loading with each incremental addition steps. The wipes were dried for 15 min post each addition step.

FIG. 6 shows calibration curve obtained with different wipe weights/sizes.

FIG. 7 shows the average cumulative amount of permeation of Lidocaine HCl in each type formulation F1-F3 (solution, wipe & ointment respectively).

FIG. 8 shows the average flux for Lidocaine HCl in each type formulation F1-F3 (solution, wipe & ointment respectively).

FIG. 9 shows the average cumulative amount of permeation of Nifedipine in each type formulation F1-F3 (solution, wipe & ointment respectively).

FIG. 10 shows the average flux for Nifedipine in each type formulation F1-F3 (solution, wipe & ointment respectively).

FIG. 11A shows average retention of Lidocaine HCl in tissue in each type formulation F1-F3 (solution, wipe & ointment respectively) the total Lidocaine HCl recovered from the tissue (amount in ng/cm2 piece); 11B shows the corresponding recovery of that applied dose of Lidocaine HCl as % recovered and 11C shows the total retention of tissue in three formulations.

FIG. 12A shows average retention of Nifedipine in tissue in each type formulation F1-F3 (solution, wipe & ointment respectively) the total Nifedipine recovered from the tissue (amount in ng/cm2 piece); 12B shows the corresponding recovery of that applied dose of Nifedipine as % recovered and 12C shows the total retention of tissue in three formulations.

FIG. 13A shows the anal canal and the various tissues that are associated with it. The topical wipe is preferably applied to tissue that is within the first 1 cm of the anal canal. The application of topical wipe by patient delivers the API (Nifedipine & Lidocaine HCl) to the external anal sphincter and aids in the treatment of anal fissures. FIG. 13B shows the skin structure of human which comprises of several layers.

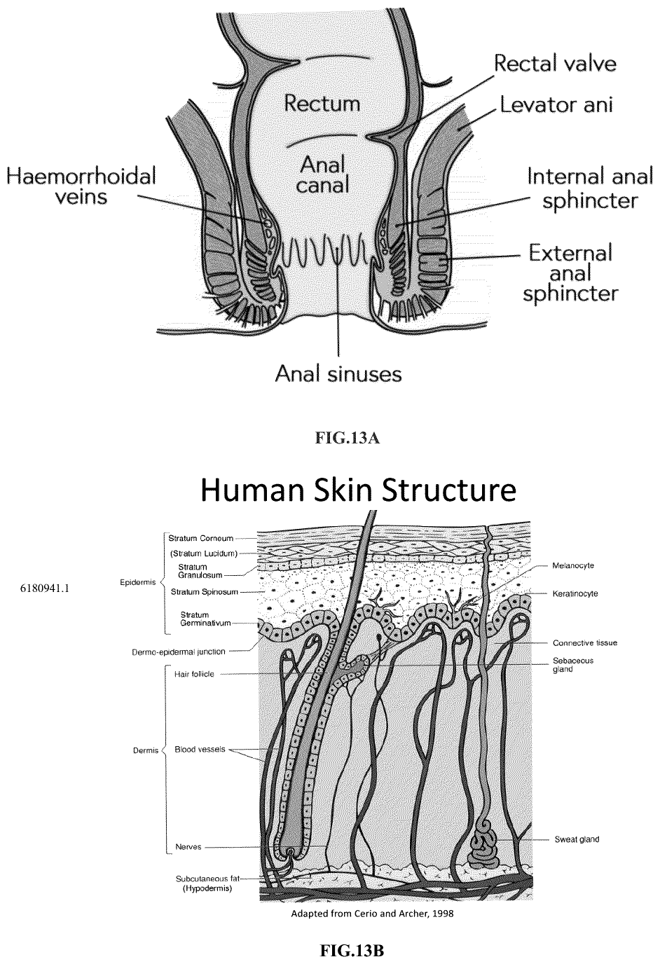

### DEFINITIONS

As used herein, the term “wipe” refers to a small to medium-sized moistened piece of plastic or cloth that either comes folded and individually wrapped for convenience or, in the case of dispenser. Wipes are generally used for cleaning purposes like personal hygiene but can be used for therapeutic purposes when impregnated with pharmaceutically active compounds such as ethyl alcohol (antiseptic wipe), or Witch Hazel (hemorrhoid wipes) etc. The topical wipes of the disclosure are impregnated with a formulation of Nifedipine (at least 0.3% w/w) and Lidocaine HCl (at least 1.5% w/w) for treatment of anal fissures. The dimension of wipes can vary depending upon the nature of use, travel sized wipes are often smaller, for example about 6 inches×3 inches. Regular wipes are for example range from about 6 inches×4 inches, from about 8 inches×8 inches, or 9 inches×7 inches. The thickness of the wipe ranges from 0.5 mm to 1 mm. Preferably the wipe is 6×4 inches and made of non-woven fiber made of cellulose.

Wipes are made of materials such as polyester, polypropylene, cotton, wood pulp, or rayon fibers formed into sheets. Wipes may be packaged individually, or in small or bulk packaging. Wipes can be made of woven fiber and non-woven fiber. Wipes are moistened with water and other inert ingredients, such as surfactants and moisturizing agents to help the active ingredient work better and to ensure user compliance. They may contain other ingredients, such as preservatives to prevent the growth of bacteria and molds. Wipes can be made biodegradable and flushable so that it is safe for septic use.

As used herein, the term “wovenfiber” refers to fiber made by interlacing two or more threads at right angles to one another. Woven fabrics or wipes can be made of both natural and synthetic fibers and are often made from a mixture of both. E.g., 100% Cotton or 80% Cotton & 20% polyester.

As used herein, the term “nonwovenfiber” refers to fiber that contain no interwoven strands but do have an organized internal structure. These are made by placing fibers together, then using heat, chemicals, or pressure to combine them into a cohesive fabric-like material. As opposed to traditional materials, such as cotton, linen, wool, and silk; non-woven fabrics do not necessitate weaving. It is made by agitating fibers in a solution until they interlock into a dense textile. Cellulose is commonly used for making nonwoven fibers.

As used herein, the term “Lidocaine” refers to the monocarboxylic acid amide resulting from the formal condensation of N,N-diethylglycine with 2,6-dimethylaniline. It acts as a local anaesthetic, and an anti-arrhythmia drug. Example of Lidocaine salt is Lidocaine HCl, other salts of Lidocaine are also contemplated to being used in topical wipes as described herein.

As used herein, the term “Lidocaine hydrochloride” or “Lidocaine HCl” or “2-(diethylamino)-N-(2,6-dimethylphenyl)acetamide;hydrochloride” refers to the hydrochloride salt from of lidocaine, an aminoethylamide and a prototypical member of the amide class anesthetics. Lidocaine interacts with voltage-gated Na+ channels in the nerve cell membrane and blocks the transient increase in permeability of excitable membranes to Na+. This prevents the generation and conduction of nerve impulses and produces a reversible loss of sensation. Lidocaine hydrochloride also exhibits class IB antiarrhythmic effects. The agent decreases the flow of sodium ions into myocardial tissue, especially on the Purkinje network, during phase 0 of the action potential, thereby decreasing depolarization, automaticity, and excitability.

As used herein, the term “Nifedipine” or “dimethyl 2,6-dimethyl-4-(2-nitrophenyl)-1,4-dihydropyridine-3,5-dicarboxylate” refers to a dihydropyridine calcium channel blocking agent. Nifedipine inhibits the transmembrane influx of extracellular calcium ions into myocardial and vascular smooth muscle cells, causing dilatation of the main coronary and systemic arteries and decreasing myocardial contractility. This agent also inhibits the drug efflux pump P-glycoprotein which is overexpressed in some multi-drug resistant tumors and may improve the efficacy of some antineoplastic agents. Nifedipine base and salts of Nifedipine are also contemplated to being used in topical wipes as described herein.

As used herein, the term “anal canal” refers to the terminal most part of the lower GI tract/large intestine, which lies between the anal verge (anal orifice, anus) in the perineum below and the rectum above.

As used herein, the term “anal fissures” refers to a cut or a tear in the thin, delicate lining of the anus. The tear often exposes the muscle around the anus, called the anal sphincter. The damage can cause that muscle to spasm, which can pull apart the edges of the fissure even more. The spasms can cause pain and slow down the healing. Bowel movements can also keep the fissures from getting better.

As used herein, the term “external anal sphincter” refers to a layer of voluntary (striated) muscle encircling the outside wall of the anal canal and anal opening. The external anal sphincter measures about 8 to 10 cm in length, from its anterior to its posterior extremity, and is about 1-2.5 cm opposite the anus, the sphincter muscle retracts on defecating. It consists of two layers: superficial and deep. The superficial layer, constitutes the main portion of the muscle, and arises from a narrow tendinous band, the anococcygeal raphe, which stretches from the tip of the coccyx to the posterior margin of the anus; it forms two flattened planes of muscular tissue, which encircle the anus and meet in front to be inserted into the central tendinous point of the perineum, joining with the superficial transverse perineal muscle, the levator ani, and the bulbospongiosus muscle also known as the bulbocavernosus. The deeper layer forms a complete sphincter to the anal canal.

As used herein, the term “internal anal sphincter” refers to a ring of smooth muscle that surrounds about the anal canal. Its inferior border is in contact with, but quite separate from, the external anal sphincter. The internal sphincter is composed of smooth muscle and is innervated by the autonomic nervous system, while the external sphincters are of striated muscle and have somatic (voluntary) innervation provided by nerves called the pudendal nerves.

As used herein, the term “perianal” or “perirectal” refers to the region surrounding the rectal orifice or the opening of the anal canal. Generally, wipes are used to clean or reach the perianal region.

As used herein, the term “intrarectal” refers to the region deep inside the rectum or the anal canal. Generally digital penetration or an applicator wand is required to access the intrarectal region.

As used herein, the term “excipient” refers to inert pharmaceutical ingredients that are used in pharmaceutical formulations. Excipients may perform a variety of functional roles in the pharmaceutical product. Each excipient serves a specific purpose (e.g., binder, disintegrant, or pH adjustment) for the proper performance of the dosage form. The properties of the final dosage form (i.e., stability) are, for the most part, highly dependent on the excipients chosen, their concentrations and interaction with both the active compound and each other. Most of the excipients used in product formulations are compendial items (United States Pharmacopeia (USP), European Pharmacopeia (EP), Japanese Pharmacopeia (JP).

As used herein, the term “API” refers to “active pharmaceutical ingredient. Any substance or combination of substances used in a finished pharmaceutical product (FPP), intended to furnish pharmacological activity or to otherwise have direct effect in the diagnosis, cure, mitigation, treatment or prevention of disease, or to have direct effect in treatment. The term API in this disclosure refers to Nifedipine and Lidocaine HCl.

As used herein, the term “preservative” refers to a substance that prevents or inhibits microbial growth and extends the shelf life of the drug products. Commonly used preservatives in these systems include sodium benzoate, EDTA, sorbic acid, and parabens.

As used herein, the term “antioxidant” refers to a substance that reduces damage of the active components due to oxidation such as that caused by free radicals. Common examples of antioxidants include butylated hydroxytoluene (BHT), alpha-tocopherol, propyl gallate, Beta Hydroxy Acids (BHA), Squalene, Ascorbyl palmitate, Sodium thiosulfate and Sodium metabisulphate.

As used herein, the term “penetration enhancer” or “permeation enhancer” refers to agents that penetrate into the skin and interact with skin constituents to promote drug flux or reversibly decrease the barrier resistance. Common examples include benzyl alcohol, dimethyl isosorbide (DMI), propylene glycol, hexylene glycol, isopropyl alcohol (IPA), ethanol, phenoxyethanol, oleic acid, olelyl alcohol, isopropyl myrisitate, medium chain triglyceride (MCT) and transcutol. Combinations of ethanol or IPA with one or more of Oleic acid, Oleyl alcohol, Medium chain triglycerides (MCT), or Isopropyl myrisate can be used as well.

As used herein, the term “moisture retainer” or “emollient” refers to a substance that forms a barrier around the surface of skin and prevents loss of moisture from skin cells. Common examples include Shea butter, Cocoa butter, Mineral oil, Lanolin, Petrolatum, Paraffin, Beeswax and Squalene.

As used herein the term “dispersing agent” is a substance that disperses another substance in a medium such as water to form a colloidal solution. Their main function is to reduce the adhesion between particles and prevent flocculation or agglomeration. Common examples include cetrimonium chloride, PEG-10 Dimethicone, PEG-7 Glyceryl cocoate and Glycereth-26.

As used herein, the term “humectant” is a hygroscopic material which absorbs water vapors and bind water to skin. Aqueous solutions of humectants can reduce the rate of loss of moisture. These are commonly added to cosmetics like oil in water type creams (vanishing cream) to prevent drying out. Common examples of humectant include propylene carbonate and Glycerin, Pentylene glycol, Butylene glycol, Aloe vera Juice/extract, Hexylene glycol, Hyaluronic acid and salts of the same, lactic acid and salts of the same.

As used herein, the term “emulsifier” is a compound or substance that acts as a stabilizer for emulsions, preventing liquids that ordinarily don't mix from separating. Common examples include Polysorbate 80, Span-80 sodium stearoyl lactylate, mono- and di-glycerols, ammonium phosphatide, PEG-40 Hydrogenated castor oil, Sodium lauryl sulfate (SLS), Poloxamers (Co block polymers), Hydrogenated castor oil, Polysorbates (20 to 80)-TWEENS, Sorbitans (SPANS (20-85), and Glyceryl monooleate (GMO).

As used herein, the term “surfactant” refers to a substance such as a detergent that, when added to a liquid, reduces its surface tension, thereby increasing its spreading and wetting properties. Surfactants have several uses in pharmaceuticals, i) for solubilization of hydrophobic drugs in aqueous media, ii) as components of emulsions, iii) surfactant self-assembly vehicles for oral and transdermal drug delivery, iv) as plasticizers in semisolid delivery systems, and v) as agents to improve drug absorption. Common examples include PEG 400, tween-80, oleyl alcohol, glycerin, hexylene glycol and propylene glycol.

As used herein, the term “viscosity modifier” refers to a substance that can change the thickness or texture of pharmaceutical ingredients. Viscosity modifiers can include such products as thickeners, texturizers, gelation agents and stiffening agents. Many viscosity modifiers can be used to convert liquids to gels, pastes or powders to aid formulators in creating the ideal product for end users. A viscosity modifier can decrease the thickness of a liquid to improve pour ability, spread ability over a surface.

As used herein, the term “filmforming agent” refers to a compound that leaves a pliable, cohesive, and continuous covering over the hair or skin when applied to their surface. This film has strong hydrophilic properties and leaves a smooth feel on skin Common examples include polyvinylpyrrolidone (PVP), acrylates, acrylamides, and copolymers. Bioadhesive polymers can be used as excipients that reduce friction between the medicated wipe and anal skin and create a film for improved retention post wiping. Common examples of such Bioadhesive polymers include Carbomer homopolymer Type A, B and C, Carbomer copolymers and interpolymers (Polycarbophils and Pemulens), Cellulose derivatives such as Hydroxyethyl cellulose (HEC), Hydroxypropyl Cellulose (HPC) and Hydroxypropyl Methylcellulose (HPMC).

As used herein the term “IVPT” refers to in vitro release testing (IVRT) entails measurement of the drug released from the vehicle into a receptor medium, separated by an inert membrane and used to quantify the amount of active pharmaceutical ingredient (API) released from semisolid dosage forms or wipes and to determine its release rate. Details on how IVPT is performed can be found in Rath S, Kanfer I. A Validated IVRT Method to Assess Topical Creams Containing Metronidazole Using a Novel Approach. Pharmaceutics. 2020; 12(2):119. Published 2020 Feb. 3. doi:10.3390/pharmaceutics12020119.

As used herein, the term “TEWL” refers to Transepidermal water loss (TEWL) is the amount of water that passively evaporates through skin to the external environment due to water vapor pressure gradient on both sides of the skin barrier and is used to characterize skin barrier function. The average TEWL in human is about 300-400 mL/day; however, it can be affected by environmental and intrinsic factors. Transepidermal water loss (TEWL) can serve as a measure of the effectiveness of the barrier properties of stratum corneum.

### Production of Wipes

The material used in topical wipes can be non-woven fabric similar to the type used in diapers and dryer sheets or woven fabric. Traditional fabrics (woven fiber) are made by weaving together fibers of silk, cotton, polyester, wool, and similar materials to form an interlocking matrix of loops which are referred to as woven fabrics. Non-woven fabrics (non-woven fiber), on the other hand, are made by a process that presses a single sheet of material from a mass of separate fibers. Fibers, such as cotton and rayon, are used in this process, as well as plastic resins like polyester, polyethylene, and polypropylene. Preferably, wipes are made of non-woven 100% cellulose (wood pulp) available from commercial sources. (https://pdicontract.com/).

Topical wipes can optionally contain mild detergents mixed with moisturizing agents, fragrance, and preservatives. The wipes can optionally contain amphoteric surfactants, such as sodium diamphoacetate and coco phosphatidyl PG-dimonium chloride used in wipes. These chemicals don't strip the skin of natural oils and also decrease skin irritation potential. Mildness is a prime consideration given that the wipe would in contact with delicate skin around the anus and genitals. Humectants such as propylene glycol and glycerine can be optionally added to prevent premature drying of the solution and contribute to skin moisturization.

In addition, oils such as mineral oil, lanolin, or silicones can be incorporated to the desired formulation (Lidocaine HCl & Nifedipine along with suitable excipients) that helps to soften skin. Thickeners, such as cellulose derivatives like hydroxymethyl cellulose, control the viscosity of the finished product and keep it the right consistency. Other ingredients include preservatives, such as methyl and propyl paraben, to ensure the solution does not support microbial growth. Fragrance can also be added to increase consumer appeal and to help over-come body odors. Natural ingredients that are known to be kind to the skin such as aloe vera or oatmeal extract can also be added to further nourish the skin.

There are two primary methods of assembling non-woven fabrics: the wet laid process and the dry laid process. A dry laid process is the “meltblown” method, which is used to make non-woven fabrics from plastic resins. In this method, plastic pellets are melted and then extruded, or forced through tiny holes, by air pressure. As the stream of fibers cools, it condenses to form a sheet. Hot metal rollers are used to flatten the fibers and bond them together.

A wet laid process is typically used for softer cloths, like diaper wipes, that use cotton blends. In this wet process, the fibers are made into liquid slurries with water and other chemicals. The resultant paste is pressed into flat sheets by rollers and then dried to form long rolls of fabric. These rolls are then further processed and slit into narrow widths and then perforated or cut into individual sheets. The finished cloths are classified by their dry weight that is at least 1.4 oz/in2 (40 g/m2). Absorbency of the wipes is also an important requirement (quality wipes can absorb between 200% and 600% of their weight in solution).

Once prepared, the non-woven cloth is fed from storage rolls onto coating machinery, where the desired formulation (Lidocaine HCl & Nifedipine along with suitable excipients) is applied. Several methods can be employed in this process. The formulation can also be added by running the fabric through a trough of the solution, or sheets of fabric may be sprayed with the formula from a series of nozzles.

Alternatively, individual towelettes may be packaged in sealed foil pouches. In this process, sheets of laminated foil are fed into automated equipment which folds them into a small pouch and heat seals three sides to form an open envelope. Simultaneously, another conveyor line feeds the non-woven cloths into the pouch. A liquid feed mechanism, including conduits extending through the stuffing bars, injects moisturizing liquid into the towelette packet simultaneously with the stuffing of the towelette material. The pouches are then heat sealed to retain wetness.

The dimension of wipes can vary depending upon the nature of use, travel sized wipes are often smaller, for example about 6 inches×3 inches. Regular wipes are for example range from about 8 inches×8 inches, or 9 inches×7 inches. The thickness of the wipe ranges from 0.5 mm to 1 mm. Wipes are made of materials such as polyester, polypropylene, cotton, wood pulp, or rayon fibers formed into sheets. Wipes may be packaged individually, or in small or bulk packaging. Wipes are moistened with water and other inert ingredients, such as surfactants and moisturizing agents to help the active ingredient work better and to ensure user compliance. They may contain other ingredients, such as preservatives to prevent the growth of bacteria and molds. Wipes can be made biodegradable and flushable so that it is safe for septic use.

### EXAMPLES

### Example 1—Solvent Screening Using Solubility Assays

Experiments were done to evaluate the visual solubility of Nifedipine and Lidocaine HCl in a series of excipients to identify suitable solvent system. A solvent system that is capable of dissolving both components was identified by this process. The solvents used for analysis are listed in Table I. An aliquot of lidocaine HCl (for example 20-30 mg) was added to each solvent contained in a scintillation vial. The vials are then closed and placed on multi-position stir plates at ambient conditions and allowed to mix for a minimum of 15 minutes. The vials are then visually inspected periodically (−1 hr) for presence or absence of turbidity, and if the drug has solubilized, additional aliquots of lidocaine HCl are to be added until saturation is achieved. The process was repeated separately using an aliquot of Nifedipine following the same protocols as described above.

The study was performed for 24 hrs with samples kept for stirring and visually inspected the following day. The final weight of each vial was taken to determine the approximate solubility in each excipient. The results of the visual solubility assay are listed in Table II.

Analysis of solubility assay indicated that Nifedipine is lipophilic in nature. Nifedipine does not easily partition into water and oil phase but can partition in solvent systems like Benzyl alcohol, DMI, Propylene carbonate, Transcutol (Diethylene glycol monoethyl ether) and Polysorbate 80. It also showed that Lidocaine HCl is hydrophilic in nature and easily partition into water and glycol phase such as Propylene glycol, Hexylene glycol, Glycerin, PEG 400, along with Benzyl alcohol, Propylene carbonate, and Transcutol (Diethylene glycol monoethyl ether).

Transcutol-P and Propylene glycol stabilized Lidocaine HCL and enhanced the wettability of dose from non-woven fabric. It also enhances the transmucosal delivery of Lidocaine HCL to the perianal tissue and improves its efficiency to serve as a local anesthetic agent. Polysorbate 80 was found to reduce the surface tension of the formulation so that the formulation comprising the active pharmaceutical component (Nifedipine & Lidocaine) is easily impregnable onto the surface of the wipe. Polysorbate 80 also enhances the delivery by increasing the wettability of the medicated wipe to the application site. Benzyl alcohol also served as a preservative of the active pharmaceutical component (Nifedipine & Lidocaine HCl). Propylene carbonate was useful for solubilizing Nifedipine. Butylated Hydroxy toluene (BHT) served as an antioxidant and stabilizer for Lidocaine HCl and Nifedipine.

TABLE 1
Excipients for Solubility Screening

| | | | |
|---|---|---|---|
| | | | FDA IID |
| | | | Limit for |
| No. | Excipients | Functional Category | Topicals |

Alcohols & Glycols

| 1. | Propylene glycol | Solvent, Permeation | 99.98% | w/w |
|---|---|---|---|---|
| | | enhancer, Emollient | | |
| 2. | Hexylene glycol | Solvent | 12.00% | w/w |
| 3. | Glycerin | Solvent | 20.00% | w/w |
| 4. | Polyethylene glycol 400 | Solvent | 69.90% | w/w |

Fatty Alcohols & Fatty acids

| | | | | |
|---|---|---|---|---|
| 5. | Oleyl alcohol | Penetration enhancer | 10.00% | w/w |
| 6. | Isopropyl myr state | Penetration enhancer, | 50.3% | w/w |

Solubilizer, emollient

Esters, ethers & Phenoxy alcohols

| | | | | |
|---|---|---|---|---|
| 7. | Benzyl alcohol | Solvent, Preservative | 2.70% | w/w |
| 8. | PPG15- stearylether | Emollient | 16.00% | w/w |
| 9. | Diethylene glycol | Solvent, Permeation | 49.91% | w/w |
| | monoethylether | enhancer | | |
| 10. | Dimethyl isosorbide | Solvent, Penetration | 15% | w/w |
| | | enhancer | | |
| 11. | Propylene Carbonate | Solvent | 5.00% | w/w |

Lipophiles-Oils

| 12. | Medium chain | Oil/Permeation | 20% | w/w |
|---|---|---|---|---|
| | triglycerides | enhancer/solubilizer | | |
| 13. | PEG40-Hydrogenated | Oil/emulsifier | 1.0% | |
| | castor oil | | | |
| 14. | Light Mineral Oil | Solvent | 39.92% | w/w |

General

| | | | | |
|---|---|---|---|---|
| 15. | Polysorbate 80 | Solubilizer | 15.00% | w/w |

| | | |
|---|---|---|
| 16. | Purified water | Solvent |

TABLE 2
Results of Solubility Assay

| API | S. No. (Solvent) | | Results (mg/g) |
|---|---|---|---|
| Nifedipine | 1. | Propylene glycol | Less than 19.33 mg/g |
| | 2. | Hexylene glycol | In between 18.07-33.69 mg/g |
| | 3. | Glycerin | Less than 15.86 mg/g |
| | 4. | Polyethylene glycol 400 | In between 33.40-50.61 mg/g |
| | 5. | Oleyl alcohol | Less than 15.55 mg/g |
| | 6. | Isopropyl myristate | Less than 13.81 mg/g |
| | 7. | Benzyl alcohol | More than 90.11 mg/g |
| | 8. | PPG15- stearylether | Less than 15.80 mg/g |
| | 9. | Diethylene glycol monoethylether | More than 91.26 mg/g |
| | 10. | Dimethyl isosorbide | In between 66-86.62 mg/g |
| | 11. | Propylene Carbonate | In between 48.92-66.53 mg/g |
| | 12. | Medium chain triglycerides | Less than 17.61 mg/g |
| | 13. | PEG40-Hydrogenated castor oil @ 37° C. | In between 16.11-30.44 mg/g |
| | 14. | Light mineral oil | Less than 15.90 mg/g |
| | 15. | Polysorbate 80 | In between 32.36-50.60 mg/g |
| | 16. | Purified water, USP | Less than 15.06 mg/g |
| Lidocaine HCl | 17. | Propylene glycol | More than 81.43 mg/g |
| | 18. | Hexylene glycol | More than 85.36 mg/g |
| | 19. | Glycerin | In between 68.45-89.46 mg/g |
| | 20. | Polyethylene glycol 400 | In between 64.69-82.66 mg/g |
| | 21. | Oleyl alcohol | In between 15.20-30.64 mg/g |
| | 22. | Isopropyl myristate | Less than 14.79 mg/g |
| | 23. | Benzyl alcohol | More than 81.98 mg/g |
| | 24. | PPG15- stearylether | Less than 15.29 mg/g |
| | 25. | Diethylene glycol monoethylether | More than 85.27 mg/g |
| | 26. | Dimethyl isosorbide | Less than 16.38 mg/g |
| | 27. | Propylene Carbonate | In between 48.24-65.08 mg/g |
| | 28. | Medium chain triglycerides | Less than 15.59 mg/g |
| | 29. | PEG40-Hydrogenated castor oil @ 37° C. | Less than 15.61 mg/g |
| | 30. | Light mineral oil | Less than 15.39 mg/g |
| | 31. | Polysorbate 80 | Less than 17.20 mg/g |
| | 32. | Purified water, USP | More than 86.99 mg/g |

### Example 2—Drug Excipient Compatibility and Stability Study

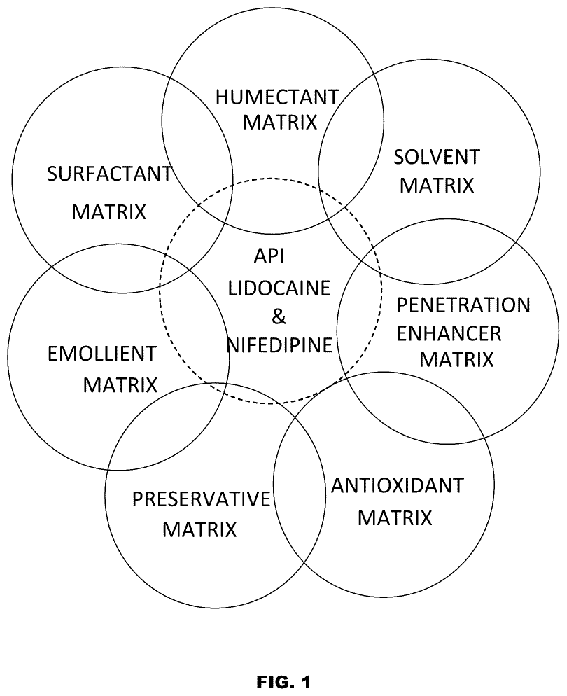

Drug-excipient compatibility studies are generally conducted with the primary goal of selecting components of dosage form that are compatible with the therapeutic components, Nifedipine and Lidocaine HCl. Various classes of excipients, such as antioxidants, penetration enhancers, moisture retainers, dispersing agents, viscosity modifiers, emollients, film forming agents, preservatives, tailing solvents, co-solvents etc. were tested in combination with the therapeutic components (Nifedipine and Lidocaine HCl) to evaluate the possibility of any incompatibilities in the formulation when exposed to excipients. FIG. 1 schematically illustrates the process of optimizing each category of excipient and the optimal concentration to arrive at formulation that contains the API (Lidocaine HCl & Nifedipine) which is then used for impregnating the wipe to create topical therapeutic wipe capable of treating anal fissures.

Most excipients have no direct pharmacological action but are important for facilitating the administration, modulating the release of the active component, and stabilizing the drug against degradation. The potential interactions between drug and excipients have effects on the chemical, physical, bioavailability and stability of the therapeutic components. Drug-Excipient mixtures were prepared, by making a solution consisting of the drug substance dissolved in a suitable solvent (Benzyl alcohol, propylene carbonate, transcutol) at a target concentration of 0.3% w/w for Nifedipine and/or Lidocaine HCl at 1.5% w/w strength.

Four sample sets were created for testing. Each sample set had 17 vials, one vial per excipient being tested and one control vial which has the active compound with no excipient. The first sample set (I) had only Nifedipine (0.3% w/w) as the therapeutic compound and each excipient shown in Table III was individually added to form the solution which was mixed and stored in a vial. The second sample set (II) had only Lidocaine HCl (1.5% w/w) as the therapeutic compound and each excipient shown in Table III was added to form the solution which was mixed and stored in a vial. The third sample set (III) had both Nifedipine (0.3% w/w) and Lidocaine HCl (1.5% w/w) as the therapeutic compound and each excipient shown in Table III was added to form the solution which was mixed and stored in a vial. The fourth sample set (IV) was a placebo control which did not have either lidocaine HCl or nifedipine but just the solvent was mixed with each excipient shown in Table III to form the solution and stored in a vial. The vials from each sample set were split into two smaller vials of equal size (A & B). Vials A from each sample set were then subjected to analysis after storage under room temperature and vials B from each sample set were subjected to analysis after storage under high temperature (40° C. & Relative Humidity of 75%) conditions. The vials were stored for 4 weeks with vials being analyzed at 2- and 4-week time periods.

TABLE 3
Excipients for DEC Nifedipine and Lidocaine HCl and their
functional categories and Maximum potency limit as per IID.

| | | | U.S. FDA IID Limits under Topical |
|---|---|---|---|
| | | | route- Maximum potency per unit |
| S. No | Ingredients | Functional Categories | dose (% w/w) |
| 1 | Nifedipine API | Active | 0.3 |
| 2 | Lidocaine HCl API | Active | 1.5 |
| 3 | Transcutol P | Solubilizer/Permeation enhancer | 49.91 |
| 4 | SR-Dimethyl isosorbide | Solubilizer/Permeation enhancer | 15 |
| | (DMI) | | |
| 5 | Benzyl Alcohol (BA) | Solubilizer/Preservative | 2.7 |
| 6 | Oleyl Alcohol (OA) | Surfactant/Emulsifier | 10 |
| 7 | Tween 80 | Surfactant/Emulsifier | 15 |
| 8 | Span 80 | Surfactant/Emulsifier | 7 |
| 9 | PEG 400 | Solvent/Plasticizer/Surfactant | 99 |
| 10 | Propylene Glycol (PG) | Vehicle/Humectant | 99.98 |
| 11 | Propylene Carbonate (PC) | Solvent/Humectant | 5 |
| 12 | Glycerin | Moisturizer/soothing | 20 |
| 13 | Hexylene Glycol | Surfactant/Emulsifier/Viscosity- | 12 |
| | | reducing agent | |
| 14 | Purified water | Solvent | 99 |
| 15 | a-Tocopherol | Antioxidant | 0.1 |
| 16 | Propyl Gallate | Antioxidant | 0.05 |
| 17 | Asorbic acid | Antioxidant | 0.3 |
| 18 | BHT | Antioxidant | 0.5 |

The A-vials in each batch were (a) monitored for visual turbidity, (b) monitored under the microscope to see signs of precipitation or crystallization and (c) pH was measured and (d) HPLC was run to see if there are any degradation product peaks (T0 measurement). The vials from each batch were then stored at room temperature (25° C. & Relative humidity of 60%) for 2 weeks and then were checked for visual turbidity, analysis under microscope and pH measurement as above (T-2 measurement). The vials from each batch were then stored at room temperature for another 2 weeks (total 4 weeks) and the same analysis was repeated (T-4 measurement). The experiment was repeated in duplicates to determine whether the excipients added are compatible with the active components (Nifedipine and Lidocaine HCl) and whether they have a stable shelf life at room temperature storage after 2-4 weeks.

The B-vials in each batch were (a) monitored for visual turbidity, (b) monitored under the microscope to see signs of precipitation or crystallization and (c) pH was measured and (d) HPLC was run to see if there are any degradation product peaks (TO measurement). The vials from each batch were then stored at high temperature (40° C. & Relative humidity of 75%) for 2 weeks and then were checked for visual turbidity, analysis under microscope and pH measurement as above (T-2 measurement). The vials from each batch were then stored at high temperature for another 2 weeks (total 4 weeks) and the same analysis was repeated. (T-4 measurement) The experiment was repeated in duplicates to determine whether the excipients added are compatible with the active components (Nifedipine and Lidocaine HCl) and whether they have a stable shelf life even when stored at high temperature conditions for 2-4 weeks.

The TO measurement of first sample set at both temperatures (RT & 40° C.) showed that the control sample (Nifedipine only) appeared to be stable at TO weeks with no impurities. No major degradation (>5%) observed in any excipient combinations at TO. The T4 measurement of first sample set at both temperatures (RT & 40° C.) showed that the control sample (Nifedipine only) appeared to be stable at T4 weeks with no impurities. Major degradation (>5%) was observed in DEC combinations of Transcutol, DMI, Benzyl Alcohol, Oleyl Alcohol, PEG 400 and Ascorbic Acid indicating incompatibilities. Slight degradation also was observed with Tween 80 combination. The degradation was markedly reduced when anti-oxidant BHT was added to the formulation.

The TO measurement of second sample set at both temperatures (RT & 40° C.) showed that the control sample (Lidocaine HCl only) appeared to be stable at TO weeks with no impurities. No major degradation (>5%) observed in any excipient combinations at TO. The T4 measurement of second sample set at both temperatures (RT & 40° C.) showed that the control sample (Lidocaine HCl only) appeared to be stable at T4 weeks with no impurities. Major degradation (>5%) was observed in DEC combinations of Transcutol, DMI, and PEG 400 combinations indicating incompatibilities. Slight degradation also was observed with Benzyl Alcohol and Oleyl alcohol combinations.

The TO measurement of third sample set at both temperatures (RT & 40° C.) showed that the control sample (Nifedipine & Lidocaine HCl only) appeared to be stable at TO weeks with no impurities. No major degradation (>5%) observed in any excipient combinations at TO. The T4 measurement of second sample set at both temperatures (RT & 40° C.) showed similar results as that of the first and second sample sets. Based on the results of the sample sets, six formulations having specific concentrations of excipients was chosen for further analysis.

### Example 3—Stability Analysis of Formulations

The following six formulations were chosen for further stability analysis.

Formulation I (F-I)

| | | FDA IID | |
|---|---|---|---|
| | | Limits | % |
| Excipients | Role | (% w/w) | w/w |
| Nifedipine | Active | NA | 0.3 |
| Lidocaine HCl | Active | NA | 1.5 |
| Benzyl Alcohol | Solubilizer/Permeation enhancer | 2.7 | 2 |
| Transcutol | Solubilizer/Permeation enhancer | 49.1 | 40 |
| Propylene | Solubilizer/Humectant | 5 | 4 |
| Carbonate | | | |
| Polysorbate 80 | Surfactant/Emulsifier | 15 | 2 |
| BHT | Antioxidant | 0.5 | 0.2 |
| PEG 400 | Solvent/Plasticizer/Surfactant | 70 | 50 |
| Total | | | 100 |

Formulation II (F-II)

| | | FDA IID | |
|---|---|---|---|
| | | Limits | % |
| Excipients | Role | (% w/w) | w/w |
| Nifedipine | Active | NA | 0.3 |
| Lidocaine HCl | Active | NA | 1.5 |
| Benzyl Alcohol | Solubilizer/Permeation enhancer | 2.7 | 2 |
| Transcutol | Solubilizer/Permeation enhancer | 49.1 | 40 |
| Propylene | Solubilizer/Humectant | 5 | 4 |
| Carbonate | | | |
| Polysorbate 80 | Surfactant/Emulsifier | 15 | 2 |
| BHT | Antioxidant | 0.5 | 0.2 |
| PEG 400 | Solvent/Plasticizer/Surfactant | 70 | 35 |
| Glycerin | Solvent/Plasticizer/Surfactant | 20 | 15 |
| Total | | | 100 |

Formulation III (F-III)

| | | FDA IID | |
|---|---|---|---|
| | | Limits | % |
| Excipients | Role | (% w/w) | w/w |
| Nifedipine | Active | NA | 0.3 |
| Lidocaine HCl | Active | NA | 1.5 |
| Benzyl Alcohol | Solubilizer/Permeation enhancer | 2.7 | 2 |
| Transcutol HP | Solubilizer/Permeation enhancer | 49.1 | 30 |
| Propylene | Solubilizer/Humectant | 5 | 4 |
| Carbonate | | | |
| Polysorbate 80 | Surfactant/Emulsifier | 15 | 2 |
| BHT | Antioxidant | 0.5 | 0.2 |
| Propylene Glycol | Solvent/Plasticizer/Surfactant | 89 | 60 |
| Total | | | 100 |

Formulation 4 (F-IV)

| | | FDA IID | |
|---|---|---|---|
| | | Limits | % |
| Excipients | Role | (% w/w) | w/w |
| Nifedipine | Active | NA | 0.3 |
| Lidocaine HCl | Active | NA | 1.5 |
| Benzyl Alcohol | Solubilizer/Permeation enhancer | 2.7 | 2 |
| Transcutol HP | Solubilizer/Permeation enhancer | 49.1 | 35 |
| Propylene | Solubilizer/Humectant | 5 | 4 |
| Carbonate | | | |
| Polysorbate 80 | Surfactant/Emulsifier | 15 | 2 |
| BHT | Antioxidant | 0.5 | 0.2 |
| Purified water | Solvent | NA | 55 |
| Total | | | 100 |

Formulation 5 (F-V)

| | | FDA IID | |
|---|---|---|---|
| | | Limits | % |
| Excipients | Role | (% w/w) | w/w |
| Nifedipine | Active | NA | 0.3 |
| Lidocaine HCl | Active | NA | 1.5 |
| Benzyl Alcohol | Solubilizer/Permeation enhancer | 2.7 | 2 |
| Transcutol HP | Solubilizer/Permeation enhancer | 49.1 | 40 |
| Propylene | Solubilizer/Humectant | 5 | 4 |
| Carbonate | | | |
| Polysorbate 80 | Surfactant/Emulsifier | 15 | 2 |
| Purified water | Solvent | NA | 40 |
| BHT | Antioxidant | 0.5 | 0.2 |
| Hexylene glycol | Solvent/Plasticizer/Surfactant | 12 | 10 |
| Total | | | 100 |

Formulation 6 (F-VI)

| | | FDA IID | |
|---|---|---|---|
| | | Limits | % |
| Excipients | Role | (% w/w) | w/w |
| Nifedipine | Active | NA | 0.3 |
| Lidocaine HCl | Active | NA | 1.5 |
| Benzyl Alcohol | Solubilizer/Permeation enhancer | 2.7 | 2 |
| Transcutol HP | Solubilizer/Permeation enhancer | 49.1 | 40 |
| Propylene | Solubilizer/Humectant | 5 | 4 |
| Carbonate | | | |
| Purified water | Solvent | NA | 20 |
| Polysorbate 80 | Surfactant/Emulsifier | 15 | 2 |
| BHT | Antioxidant | 0.5 | 0.2 |
| Propylene Glycol | Solvent/Plasticizer/Surfactant | 89 | 30 |
| Total | | | 100 |

Each formulation was tested at three different temperatures conditions (25° C./RH 60%; 30° C./RH 65%; 40° C./RH 75%) and four different storage time points (TO, 1 month, 2 months & 3 months). The formulation was then (a) monitored for visual turbidity, (b) monitored under the microscope to see signs of precipitation or crystallization and (c) pH was measured and (d) HPLC was run to see if there are any degradation product peaks after each time point as noted in Example 2.

T-0 measurements indicated that all six formulations were stable and no degradation profiles were observed on the HPLC even when exposed to different temperatures. (25° C., 30° C. and 40° C.).

The T-1 month measurements indicated that all formulations except formulation −1 were stable. In F-I formulation, three impurities (RRT 0.293, RRT 2.630 and RRT 2.796) were observed at ˜0.75% total which were not observed in any other formulations.

The T-2-month measurements indicated that F-I formulation had exhibited some degradation. In F-I formulation, three impurities (RRT 0.293, RRT 2.630 and RRT 2.796) were observed at ˜1.1% total at 40° C. and ˜0.5% at 25° C., which were not observed in any other formulations. Likewise, F6 formulations had demonstrated an impurity at RRT 2.672 at 0.1-0.2% total at 40° C.

The T-3-month measurements indicated that F-I and F-II formulation had exhibited some degradation. The impurity at RRT 0.106 observed in F-I and F-II formulations remained consistent with previous timepoint. In F-I formulation, three impurities (RRT 0.293, RRT 2.630 and RRT 2.796) were observed at ˜1.4% total at 40° C. and ˜0.8% at 25° C., which were not observed in any other formulations. F-III formulation has no impurities observed. F-IV, F-V and F-VI formulations had demonstrated no major increase in impurities from previous timepoint.

### Example 4—Saturation Loading Capacity of Wipes

Formulation III was chosen to be tested on its ability to be loaded onto a wipe. The protocols described in this example can be applied to any formulation disclosed herein. A placebo solution using formulation composition 3 without active substances was used for the purpose of establishing saturation loading content.

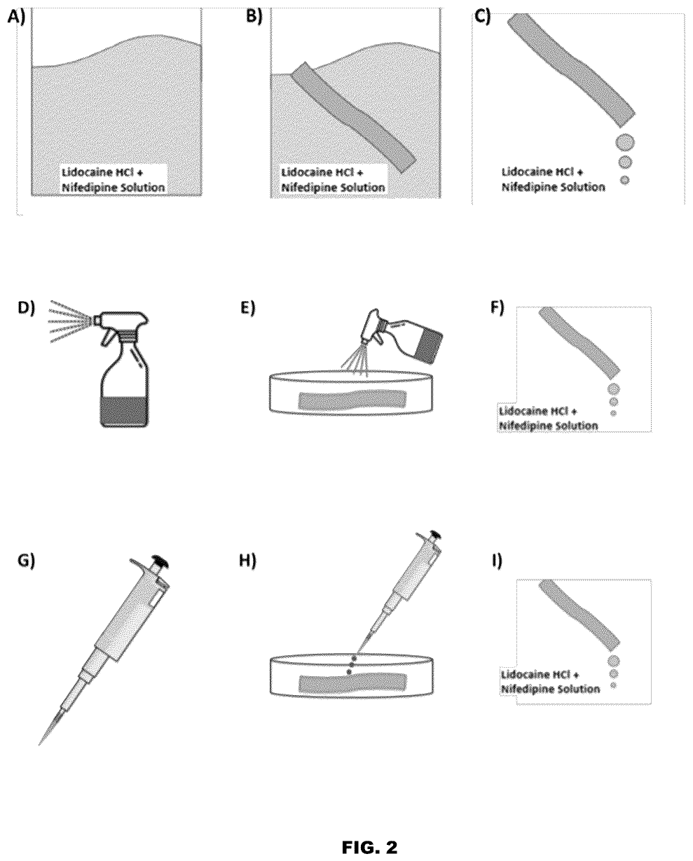

As depicted in FIG. 2, the formulation was added to the wipes either by dipping, pipetting, or spraying techniques.

### Dipping

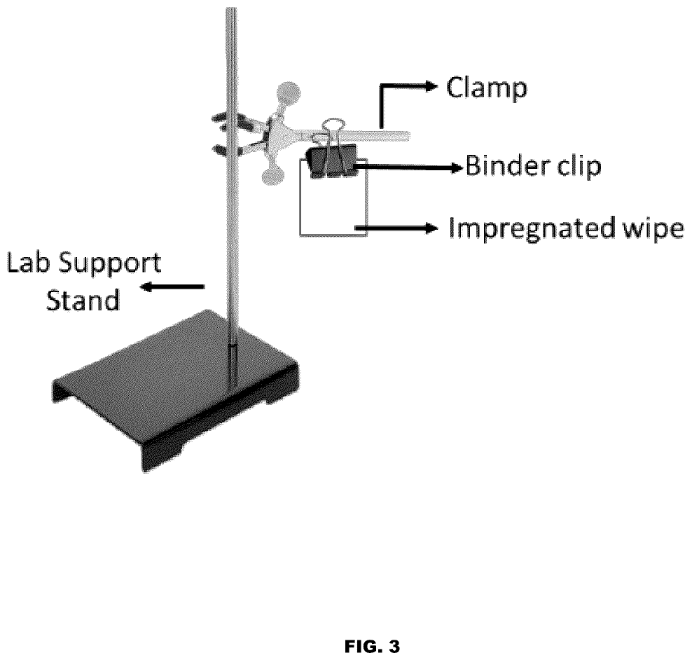

For dipping, tare dry weight of the wipe of approximately 6×4 inches was taken in a tared container and then the wipe was added to a container which contained approximately 30 g of placebo formulation during each time of addition. The wipes were immersed in the formulation for approximately 1 minute, after which the wipes were dried as depicted in FIG. 3. Each drying step took approximately 15 min. The drying time was estimated based on the amount of dripping, at 15 min almost no dripping was noticed, and this was assumed as a point at which at least 50% of drying occurred. Now, the wipe was weighed again to establish the dried weight of the wipe post one formulation addition step.

### Pipetting

For pipetting, tare dry weight of the wipe of approximately 6×4 inches was taken in a tared container and then the wipe was pipetted with approximately 5 g of placebo formulation during each time of addition. The 5 g addition was determined based on a previous study, where more than 5 g formulation addition each time showed dripping. The wipes were immersed in the formulation for approximately 1 minute, after which the wipes were dried as depicted in the FIG. 3. Now, the wipe was weighed again to establish the dried weight of the wipe post one formulation addition step.

### Spraying

For spraying, tare dry weight of the wipe of approximately 6×4 inches was taken in a tared container and then the wipe was sprayed with approximately 5 g of placebo formulation during each time of addition using a spray bottle. The wipes were immersed in the formulation for approximately 1 minute, after which the wipes were dried as depicted in FIG. 3. Now, the wipe was weighed again to establish the dried weight of the wipe post one formulation addition step.

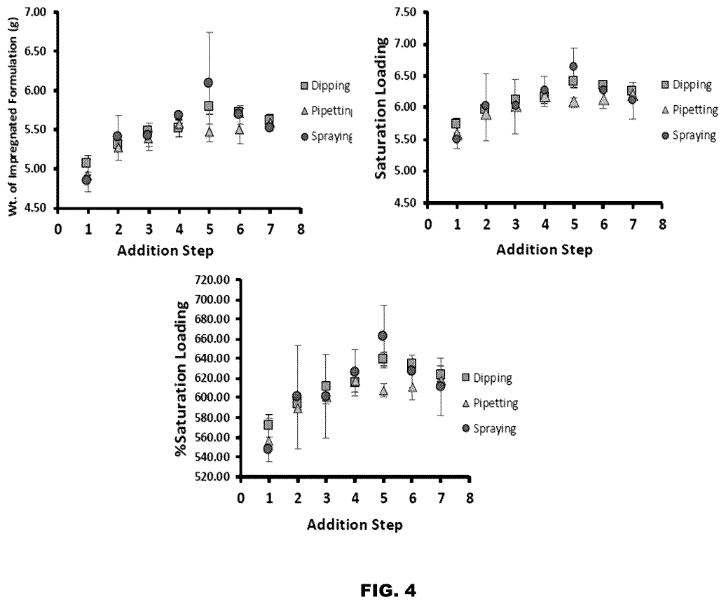

The study has demonstrated that the drying time, number of additions, and the application process do not play a significant role in controlling or limiting the saturation ability of the wipes with the formulation. The table below and figures (FIGS. 4 & 5) gives the details of the study and their respective calculations for weight of impregnated formulation, saturation loading and % saturation loading.

FIG. 4 shows that the weight of the impregnated formulation ranged between 4.8 g and 6.05 g with no trend followed with respect to the number of additions or application process. The wipes were allowed to dry for over 45 minutes prior to weighing. Saturation loading was found to be between 5.5 and 6.5 with no trend followed with respect to the number of additions or application process. Percent saturation loading was found to range from 550 to 650 with no trend followed with respect to the number of additions or application process. Thus, the results demonstrated that the number of additions and the application process do not have any impact on the amount of formulation impregnated or the saturation loading of the wipes.

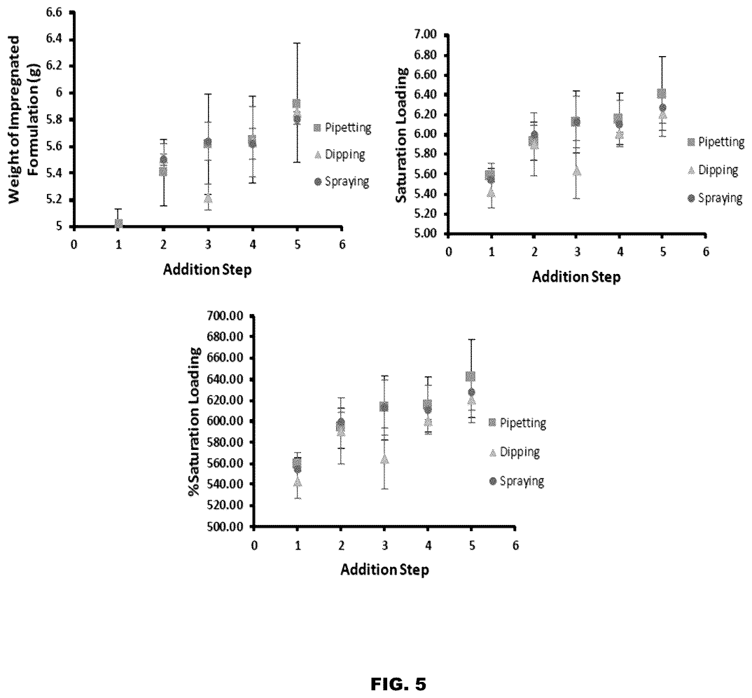

The same experiment was repeated with an increased drying time of 15 mins. The results of the experiment are shown in FIG. 5. As in the case of 45 min drying time, the weight of the impregnated formulation ranged between 3.6 g and 5.8 g with no trend followed with respect to the number of additions or application process. Saturation loading was found to be between 4.2 and 6.2 with no trend followed with respect to the number of additions or application process. Percent saturation loading was found to range from 420 to 620 with no trend followed with respect to the number of additions or application process. Thus, the results demonstrated that the number of additions and the application process do not have any impact on the amount of formulation impregnated or the saturation loading of the wipes.

Further to establish the uniformity in loading a calibration study was performed by using different weights and sizes of wipes and dipping them in the formulation and subjecting them to the same process parameters. Weights of the wipes were measured pre and post loading with formulation. The Table below shows the results obtained.

TABLE 4
Saturation loading study resulting in weight of impregnated formulation, saturating loading, %
saturation loading study and its respective calculations for 15 min drying time post each formulation additional step.

| | Wipe-1 | | | | Wipe-2 | | | | | | | | | | |
|---|---|---|---|---|---|---|---|---|---|---|---|---|---|---|---|
| | Weight of | | | | Weight of | | | | | | | | | | |
| | Impreg- | | | [symbol] | Impreg- | | | [symbol] | | | | | | | |
| | nated | | Sat- | Sat- | nated | | Sat- | Sat- | | Weight of | | | | [symbol] | |
| | Formu- | [symbol] | uration | uration | Formu- | [symbol] | uration | uration | Addi- | Impregnated | | Saturation | | Saturation | |
| | lation | of Wipe | Load- | Load- | lation | of Wipe | Load- | Load- | tion | Formulation | | Loading | | Loading | |
| | ( [symbol] ) | ( [symbol] ) | ing | ing | ( [symbol] ) | ( [symbol] ) | ing | ing | Step | Mean | Stdev | Mean | Stdev | Mean | Stdev |
| Dipping | [symbol] | 1. [symbol] | [symbol] | [symbol] | [symbol] | 1.0 [symbol] | [symbol] | [symbol] | 1 | [symbol] | 0. [symbol] | [symbol] | 0. [symbol] | [symbol] | [symbol] |
| | [symbol] | | [symbol] | [symbol] | [symbol] | | [symbol] | [symbol] | 2 | [symbol] | 0. [symbol] | [symbol] | 0. [symbol] | [symbol] | [symbol] |
| | [symbol] | | [symbol] | [symbol] | [symbol] | | [symbol] | [symbol] | 3 | [symbol] | 0. [symbol] | [symbol] | 0. [symbol] | [symbol] | [symbol] |
| | [symbol] | | [symbol] | [symbol] | [symbol] | | [symbol] | [symbol] | 4 | [symbol] | 0. [symbol] | [symbol] | 0. [symbol] | [symbol] | [symbol] |
| | [symbol] | | [symbol] | [symbol] | [symbol] | | [symbol] | [symbol] | 5 | [symbol] | 0. [symbol] | [symbol] | 0. [symbol] | [symbol] | [symbol] |
| | [symbol] | | [symbol] | [symbol] | [symbol] | | [symbol] | [symbol] | 6 | [symbol] | 0. [symbol] | [symbol] | 0. [symbol] | [symbol] | [symbol] |
| | [symbol] | | [symbol] | [symbol] | [symbol] | | [symbol] | [symbol] | 7 | [symbol] | 0. [symbol] | [symbol] | 0. [symbol] | [symbol] | [symbol] |
| [symbol] | [symbol] | 1.0 [symbol] | [symbol] | [symbol] | [symbol] | 1.0 [symbol] | [symbol] | [symbol] | 1 | [symbol] | 0. [symbol] | [symbol] | 0. [symbol] | [symbol] | [symbol] |
| | [symbol] | | [symbol] | [symbol] | [symbol] | | [symbol] | [symbol] | 2 | [symbol] | 0. [symbol] | [symbol] | 0. [symbol] | [symbol] | [symbol] |
| | [symbol] | | [symbol] | [symbol] | [symbol] | | [symbol] | [symbol] | 3 | [symbol] | 0. [symbol] | [symbol] | 0. [symbol] | [symbol] | [symbol] |
| | [symbol] | | [symbol] | [symbol] | [symbol] | | [symbol] | [symbol] | 4 | [symbol] | 0. [symbol] | [symbol] | 0. [symbol] | [symbol] | [symbol] |
| | [symbol] | | [symbol] | [symbol] | [symbol] | | [symbol] | [symbol] | 5 | [symbol] | 0. [symbol] | [symbol] | 0. [symbol] | [symbol] | [symbol] |
| | [symbol] | | [symbol] | [symbol] | [symbol] | | [symbol] | [symbol] | 6 | [symbol] | 0. [symbol] | [symbol] | 0. [symbol] | [symbol] | [symbol] |
| Spraying | [symbol] | 1. [symbol] | [symbol] | [symbol] | [symbol] | 1.0 [symbol] | [symbol] | [symbol] | 1 | [symbol] | 0. [symbol] | [symbol] | 0. [symbol] | [symbol] | [symbol] |
| | [symbol] | | [symbol] | [symbol] | [symbol] | | [symbol] | [symbol] | 2 | [symbol] | 0. [symbol] | [symbol] | 0. [symbol] | [symbol] | [symbol] |
| | [symbol] | | [symbol] | [symbol] | [symbol] | | [symbol] | [symbol] | 3 | [symbol] | 0. [symbol] | [symbol] | 0. [symbol] | [symbol] | [symbol] |
| | [symbol] | | [symbol] | [symbol] | [symbol] | | [symbol] | [symbol] | 4 | [symbol] | 0. [symbol] | [symbol] | 0. [symbol] | [symbol] | [symbol] |
| | [symbol] | | [symbol] | [symbol] | [symbol] | | [symbol] | [symbol] | 5 | [symbol] | 0. [symbol] | [symbol] | 0. [symbol] | [symbol] | [symbol] |
| | [symbol] | | [symbol] | [symbol] | [symbol] | | [symbol] | [symbol] | 6 | [symbol] | 0. [symbol] | [symbol] | 0. [symbol] | [symbol] | [symbol] |
| | [symbol] | | [symbol] | [symbol] | [symbol] | | [symbol] | [symbol] | 7 | [symbol] | 0. [symbol] | [symbol] | 0. [symbol] | [symbol] | [symbol] |

[symbol]

indicates data missing or illegible when filed

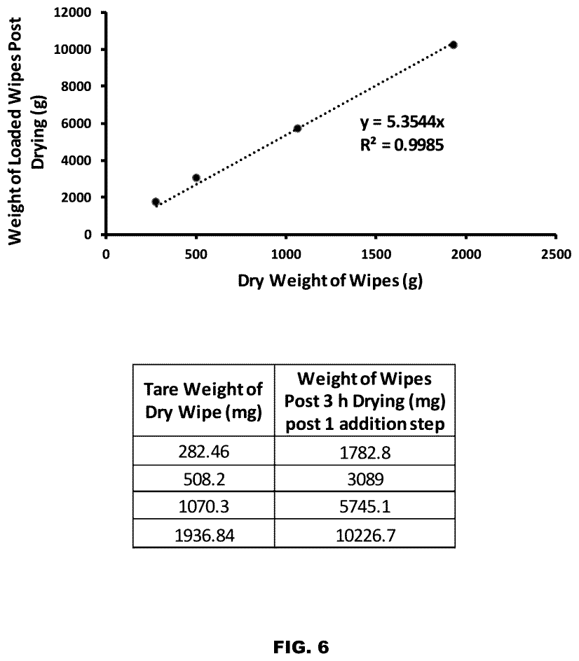

Calibration curve in FIG. 6 shows that the loading is consistent with different wipe sizes indicating that the formulation is uniformly distributed in the wipe.

### Example 5—In Vitro Permeation Testing of Therapeutic Wipe

In Vitro Permeation Testing (IVPT) was designed to determine the in-vitro permeation profile of a specific active pharmaceutical ingredient (API), formulated in a well-defined topical product, into the targeted human tissue. The permeation profile of a specific API is determined not only by the physical and chemical properties of the API (such as size, solubility, lipophilicity, log P, etc.) but is also influenced by the microstructure characteristics (viscosity, selected permeation enhancers, emulsifiers, etc.) and dosage form of the topical product. Thus, IVPT is an important tool which provides information and understanding not only regarding the feasibility of the Nifedipine and Lidocaine HCl together in single formulation, but also demonstrates the effects of various formulation prototypes on the ability of the API to reach the intended targeted site of action.

The protocols for IVPT testing are disclosed in Santos et al. (Santos L L, Swoford N J, Santiago B G. In Vitro Permeation Test (IVPT)for Pharmacokinetic Assessment of Topical Dermatological Formulations. Curr Protoc Pharmacol. 2020 December; 91(1):e79.)

In vitro permeation testing (IVPT) was done to compare the skin penetration of topical solution comprising the formulation III, its corresponding solution on wipe, and an ointment formulation containing Lidocaine HCl, 1.5% and Nifedipine, 0.3% across ex vivo human cryopreserved cadaver perianal tissue. The Experiments can be repeated with other formulations (I-II & IV-VI) following the same procedures disclosed herein.

Reagents and Materials
a) Lidocaine HCl, 1.5% & Nifedipine, 0.3% formulation III
   i. Formulation III as a Solution, (F1)
   ii. Formulation III impregnated on a wipe (following protocols in Example 4) (F2)
   iii. Formulation III as an Ointment (F3)
b) Cryopreserved human cadaver perianal tissue (500±250 μm, thickness),
c) 4 replicates per formulation was used
d) 1× Phosphate buffer saline (PBS, pH 7.4)
e) Homogenizing solution (50/50 methanol/LCMS grade water)
f) 20-mL scintillation vials
g) 6.0-mL homogenization vials with ceramic beads
h) 96-well plate for mass spec analysis
i) Wash solvent (70% v/v ethanol)

Equipment
a) Automated, Vertical in-Line Flow-Through Cells (PermeGear Collector FC 33, Version 3.1)
b) Positive displacement pipette
c) Caliper/Measuring gauge
d) Vapometer (Delfin Technologies)
e) Infrared/laser thermometer
f) Probe thermometer
g) Tissue homogenizer
h) 60° C. lab oven
i) Mass-spec
j) Centrifuge

The cryopreserved, human cadaver perianal tissue was obtained from commercial sources and stored upon arrival at −80° C. The skin tissue was thawed at room temperature and was inspected for any visible holes, damage, extensive stretching, or scarring. Trans epidermal Water Loss (TEWL or TWL) is commonly used for characterizing skin tissue barrier function. The TEWL value for each skin tissue sample was measured to estimate tissue integrity using a Vapometer. A low TEWL value is generally a characteristic feature of an intact tissue barrier function. The TEWL for each diffusion cell (Franz Diffusion Cell) is measured and recorded before application of the formulation (F1-F3).

An automated in-line flow through diffusion cell system (PermeGear Collector FC 33, Version 3.1) was used to assess drug/API in the tissue permeation experiment. An ambient environmentally controlled laboratory temperature range of 21° C.±2° C. and a humidity range of 50%±20% relative humidity was maintained throughout the IVPT study. The flow through diffusion cell system was equilibrated with receptor media.

The heater and circulator were adjusted so that the skin sample is maintained at 32° C. 1° C. The diffusion cells were placed and heated in support of the moving arm of the system to maintain the skin surface temperature (at 32° C.±1° C.). The cells were connected to multi-channel peristaltic pumps and the outlet of the diffusion cells were directed to drip into 20 mL scintillation vials. Sections of skin tissue were cut into—1.5×1.5 cm sections and mounted in the diffusion cells, with epidermal layers/stratum corneum side up. Donor compartment blocks were placed on the skin tissue (donor chamber on stratum corneum) and secured using stainless steel clamps to provide a leak proof seal. Air bubbles were removed by inverting diffusion cells and confirmed visually by glass windows on the underside of the diffusion cells. The pumps were adjusted to maintain a flowrate of −30 μL/min (1.8 mL/h) to provide adequate sink conditions.

The diffusion cells were allowed to equilibrate for approximately 30 min. Any cell showing water accumulation on top of the tissue was removed from analysis. After equilibration, an infrared or appropriate thermometer was used to measure the temperature of each cell. The skin surface temperature was maintained stable at 32° C. 1° C. The T ‘0’ samples were collected before dosing for 30 mins at −30 μL/min (1.8 mL/h).

Each formulation (F1, F2, F3) was uniformly dispensed in an alternating sequence on successive diffusion cells onto the skin tissue surface using a positive displacement pipette, set to deliver appropriately 10 μL volume. The donor chambers were kept close to ambient conditions. Fractions are collected at intervals as indicated in the protocol for up to 24-hour time point. At the end of the study, the undosed skin samples were collected followed by treated skin.

The washed and tape stripped skin was placed on aluminum foil with dermis side down. The perianal tissue samples if applicable is placed in an oven set at 60° C. for approximately 2.0 minutes. Tweezers were used to separate the epidermis from the dermis. The cleaned tissue was placed in respective pre-weighed labeled vials and weight of tissues were recorded. The undosed skin samples were processed first and placed in vial, followed by treated skin.

The cleaned epidermis and dermis tissues were placed in respective pre-weighed labeled vials and weight of tissues are recorded. Epidermis and dermis tissue samples were homogenized using Omni Prep homogenizer following the protocol below:
a) Tissue was minced appropriately and uniformly using fresh surgical blade.
b) 2000 μL of homogenizing solvent (50:50, v/v methanol/LC-MS grade Water) was added to the labelled tubes containing the skin samples.
c) Tissue was homogenized at 4.5 m/s, 3×30 seconds, 45 seconds pause period.
d) After homogenization is completed, 200 μL of this aliquot was used for API (nifedipine & lidocaine HCl) extraction using 800 μL of acetonitrile, vortexed briefly, then sonicated for 10 minutes in water bath
e) Vials are centrifuged at 10,000 RPM for 5 minutes at 5° C.
f) Supernatant will be used for mass spectrometry analysis to confirm the presence of and quantify (LC/MS) the amount of API.

Recovery of formulation on the surface of the skin tissue was performed by cleaning with 1 dry cotton swab, 1 wet cotton swab dipped in wash solvent of 70% ethanol (v/v), and 1 dry cotton swab followed by one consecutive tape strip. Swabs and tape strips were collected in labeled vials for analysis. The tissue was then subjected to homogenization and protein crash to extract the adsorbed formulation. The undosed skin samples were first cleaned followed by treated skin on a clean dissection board.
a) 10 mL ACN was added to the tapes and swabs samples collected, placed on a stir plate, and stirred overnight at 600 RPM.
b) 200 μL aliquot of the supernatant was be used for mass spectrometry analysis.

All samples were stored at 2-8° C. for short-term storage (less than a week) or −20° C. for long-term storage (more than a week) until analysis by LC-MS/MS. Analysis indicated that the cumulative amount of permeation for Lidocaine HCl through the tissue was higher for solution-based delivery formulation (F1) when compared with that of wipe-based delivery formulation (F2) and ointment-based delivery formulation (F3).

The formula for calculating the cumulative amount of permeation is the concentration of analyte x volume of sample collected at each time which is then is added to the same collect at the previous time point. Jmax is the calculated maximum flux value achieved in the study duration by each replicate. The formula to calculate Jmax mathematically is partition coefficient, Kp expressed in cm/h which is the diffusion characteristic of the analyte multiplied by the water saturation solubility Csat-max expressed as mg/L. Jmax=Kp×Csat-max.

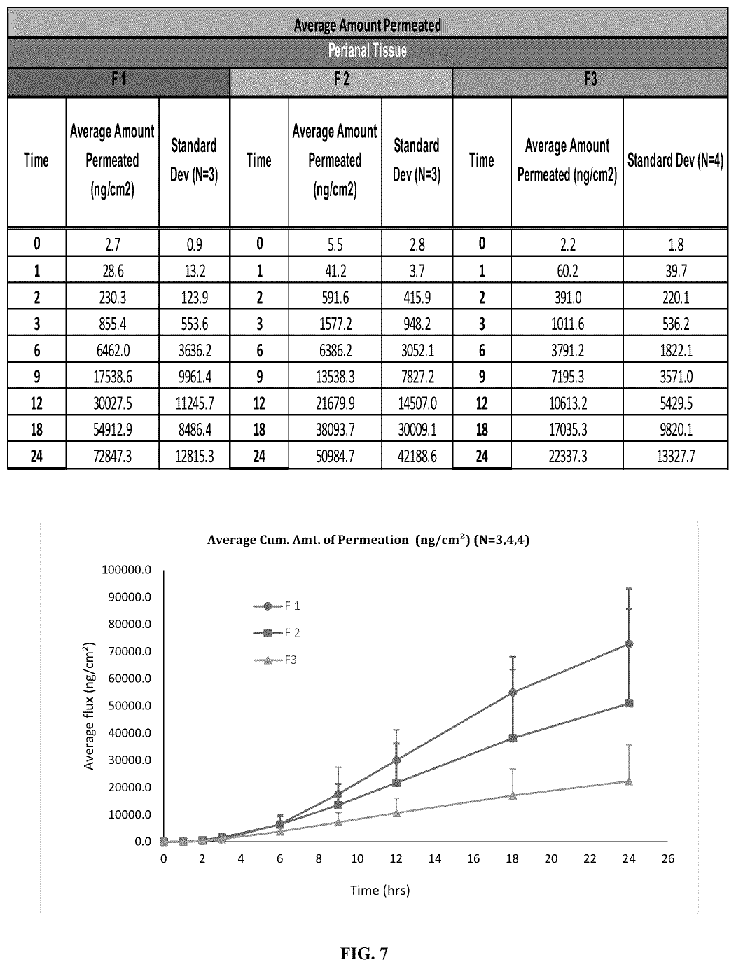

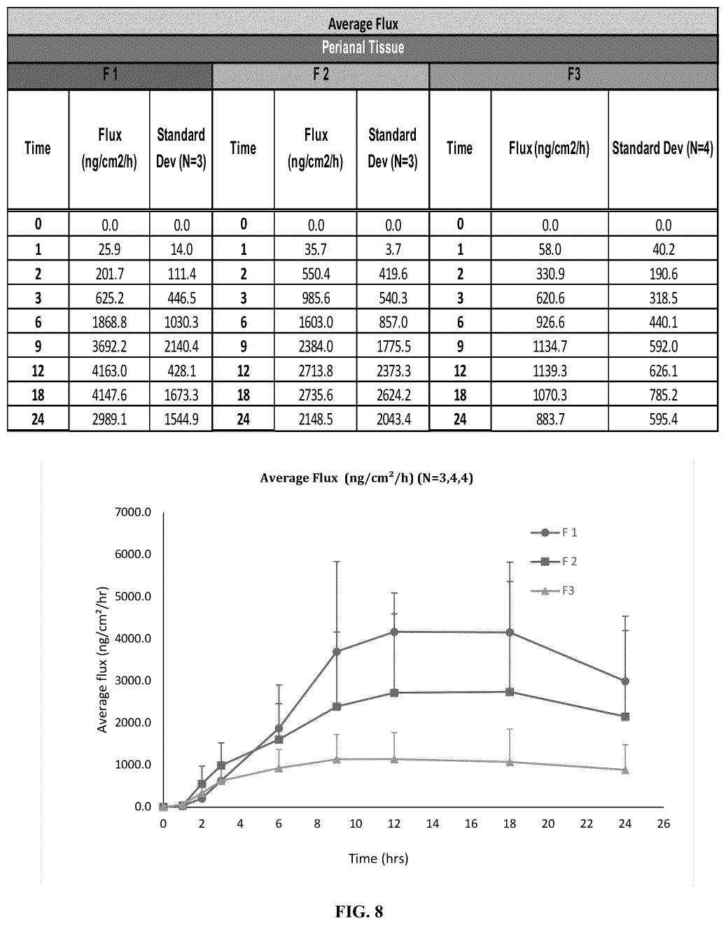

Interestingly, the cumulative amount of permeation for Lidocaine HCl through the tissue was higher for wipe-based delivery formulation (F2) when compared with that of ointment-based delivery formulation (F3). (See FIG. 7). Similar trends were seen in the case of average flux measurement which was higher in F1 when compared with F2 and F3. Likewise, the average flux for F2 was higher than that of F3. (See FIG. 8).

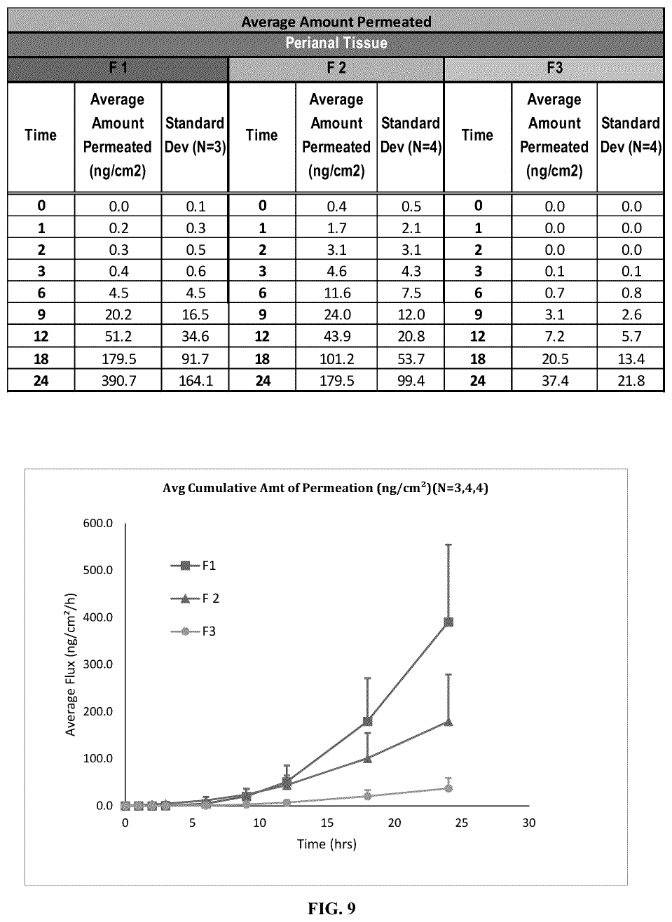

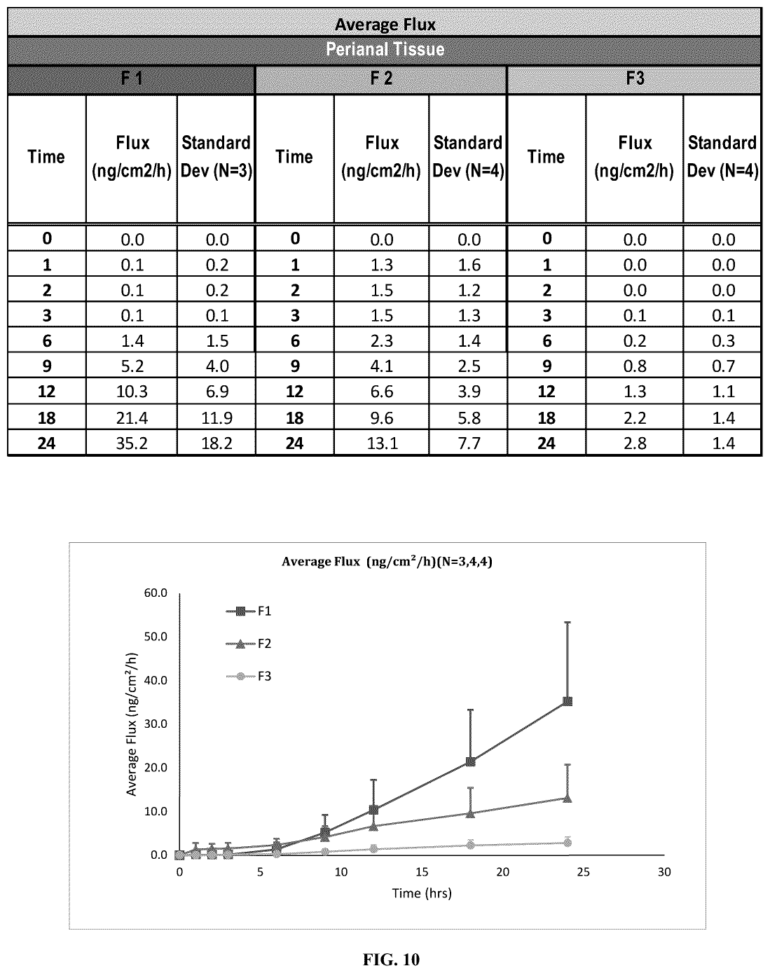

Analysis also indicated that the cumulative amount of permeation for Nifedipine through the tissue was higher for solution-based delivery formulation (F1) when compared with that of wipe-based delivery formulation (F2) and ointment-based delivery formulation (F3). Interestingly, the cumulative amount of permeation for Nifedipine through the tissue was higher for wipe-based delivery formulation (F2) when compared with that of ointment-based delivery formulation (F3). (See FIG. 9). Similar trends were seen in the case of average flux measurement which was higher in F1 when compared with F2 and F3. Likewise, the average flux for F2 was higher than that of F3. (See FIG. 10).

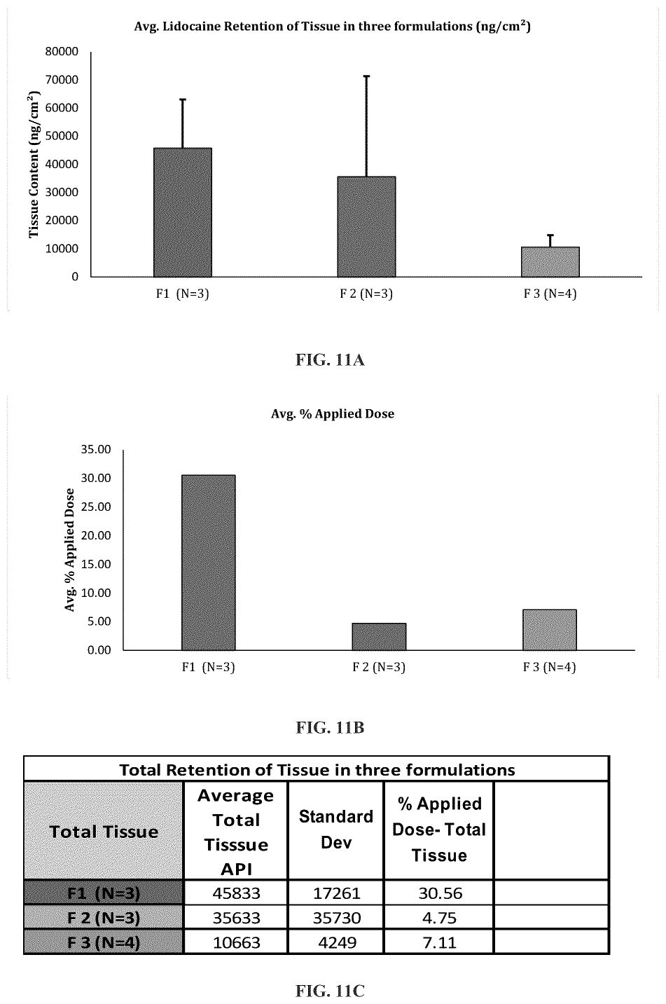

Lidocaine HCl retention in tissues for three types of delivery formulations is shown in FIG. 11. The retention of Lidocaine HCl in tissue was higher in solution-based delivery formulation (F1) when compared with that of wipe-based delivery formulation (F2) and ointment-based delivery formulation (F3). Likewise, the retention of Lidocaine HCl in tissue was higher in wipe-based delivery formulation (F2) than the ointment-based delivery formulation (F3).

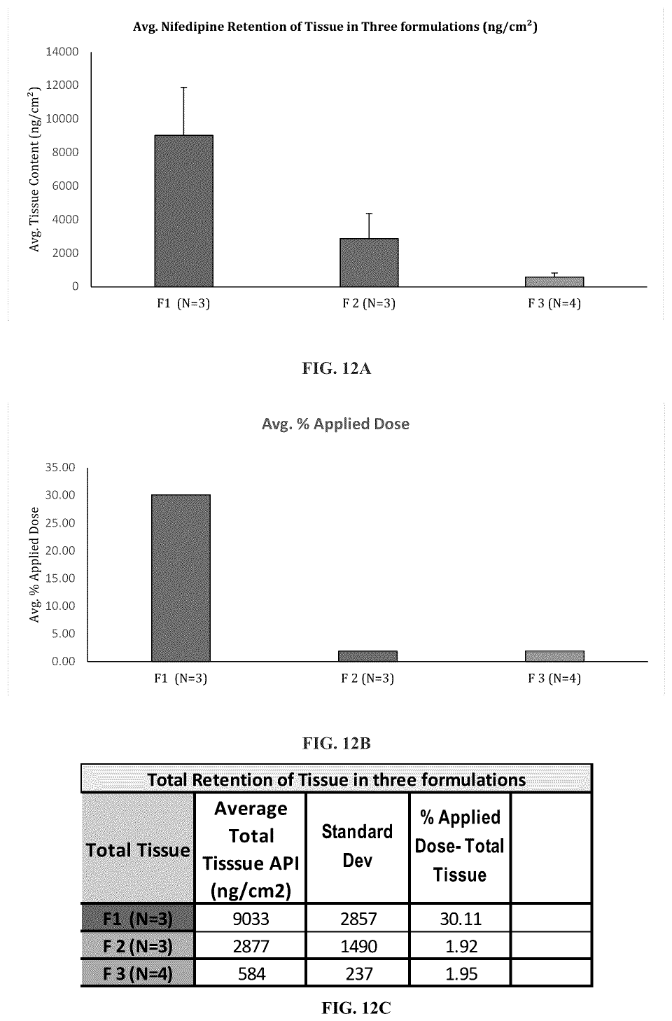

Nifedipine retention in tissues for three types of delivery formulations is shown in FIG. 12. The retention of Nifedipine in tissue was higher in solution-based delivery formulation (F1) when compared with that of wipe-based delivery formulation (F2) and ointment-based delivery formulation (F3). Likewise, the retention of Nifedipine in tissue was higher in wipe-based delivery formulation (F2) than the ointment-based delivery formulation (F3).

Experiments showed that wipe-based delivery is more effective than ointment based delivery of Nifedipine & Lidocaine HCl. The formulation of Nifedipine & Lidocaine HCl delivered by means of wipes was able to penetrate for up to 24 hours through the perianal tissue and was observed by IVPT studies. Topical solution presents absolute availability of the drug molecule at the site of action but is not practical for application from the perspective of patient use and compliance.

### INCORPORATION BY REFERENCE

All publications and patents mentioned herein are hereby incorporated by reference in their entirety as if each individual publication or patent was specifically and individually indicated to be incorporated by reference.

### OTHER EMBODIMENTS

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1-50. (canceled)

51. A topical wipe comprising a fibrous substrate, wherein said fibrous substrate comprises 0.15% to 7.5% w/w lidocaine by concentration and 0.15% to 1.5% w/w nifedipine by concentration.

52. The topical wipe of claim 51, wherein said fibrous substrate comprises 1.5% to 5% w/w lidocaine by concentration and 0.3% to 1% w/w nifedipine by concentration.

53. The topical wipe of claim 52, wherein said wipe further comprises at least one excipient.

54. The topical wipe of claim 52, wherein said wipe further comprises an antioxidant.

55. The topical wipe of claim 52, wherein said wipe further comprises a preservative.

56. The topical wipe of claim 52, wherein said wipe further comprises an alcohol solvent.

57. The topical wipe of claim 52, wherein the wipe further comprises a solvent selected from the group consisting of water, propylene glycol and hexylene glycol.

58. The topical wipe of claim 52, wherein said wipe further comprises one or more of benzyl alcohol, transmute, propylene carbonate, polysorbate 80, BHT, PEG 400, glycerin and hexylene glycol.

59. The topical wipe of claim 52, wherein said wipe is made from a fiber.

60. The topical wipe of claim 53, wherein said excipient is selected from the group consisting of: antioxidants, penetration enhancers, preservatives, moisture retainers, dispersing agents, humectant, emulsifier, plasticizer, surfactant, polymer, viscosity modifiers, emollients, film forming agents, tailing solvents, co-solvents, and/or oils.

61. The topical wipe of claim 60, wherein said penetration enhancer is selected from the group consisting of benzyl alcohol, dimethyl isosorbide (DMI), propylene glycol, hexylene glycol, isopropyl alcohol, ethanol, phenoxyethanol, oleic acid, olelyl alcohol, isopropyl myrisitate, medium chain triglyceride (MCT) and transcutol.

62. The topical wipe of claim 60 wherein said humectant is selected from the group consisting of propylene carbonate, glycerin, pentylene glycol, butylene glycol, aloe vera juice, extract, hexylene glycol, hyaluronic acid and lactic acid.

63. The topical wipe of claim 60, wherein said emulsifier is selected from the group consisting of Polysorbate (20 to 80), Span-80, PEG-40, hydrogenated castor oil, sodium lauryl sulfate (SLS), poloxamers, sorbitans (20-85) and glyceryl monooleate (GMO).

64. The topical wipe of claim 60, wherein said surfactant is selected from the group consisting of PEG 400, tween-80, oleyl alcohol, glycerin, hexylene glycol and propylene glycol.

65. The topical wipe of claim 60, wherein said antioxidant is selected from the group consisting of butylated hydroxytoluene (BHT), alpha-tocopherol, propyl gallate, ascorbic acid squalene, ascorbyl palmitate, sodium thiosulfate and sodium metabisulphate.

66. The topical wipe of claim 60, wherein said polymer is selected from the group consisting of carbomer homopolymer Type A, B and C, carbomer copolymers, interpolymers, polycarbophils, pemulens, hydroxyethyl cellulose (HEC), hydroxypropyl cellulose (HPC) and hydroxypropyl methylcellulose (HPMC).

67. A container comprising a plurality of topical wipe of claim 51.

68. The container of claim 67, wherein said container comprises a soft pack or a hard pack.

69. A method of treating or ameliorating anal fissures in a subject in need thereof, comprising the step of applying the topical wipe of claim 51 to the perianal area of said subject thereby providing therapeutic relief.

70. The method of claim 69, wherein the topical wipe is applied to anal tissue within 1 cm from the anal canal of said subject thereby providing therapeutic relief.

71. The method of claim 69, wherein the topical wipe is applied to an external anal sphincter of said subject thereby providing therapeutic relief.

72. A method of manufacture of topical wipe comprising 0.3-1% nifedipine by concentration and 1.5%-5% lidocaine by concentration, comprising the steps of: providing a wipe, applying a formulation comprising at least 0.3% nifedipine and at least 1.5% lidocaine, drying said wipe for sufficient time and packaging said wipe in an airtight container or pouch.
